(19) United States
(12) Patent Application Publication
(10) Pub. No.: US 2002/0016974 A1
(43) Pub. Date: Feb. 7, 2002

(54) Transgenic, non-human animals containing a coxsackie/adenovirus receptor (CAR)

(76) Inventors: Robert T. Woodland, Worcester, MA (US); Madelyn R. Schmidt, North Grafton, MA (US); Brian Piekos, Boston, MA (US)

Correspondence Address: J. PETER FASSE, Fish & Richardson P.C., 225 Franklin Street, Boston, MA, 02110-2804, US

(21) Appl. No.: 09/788,025

(22) Filed: Feb. 16, 2001

Related U.S. Application Data
(60) Provisional application No. 60/183,080, filed on Feb. 16, 2000.

(51) Int. Cl. A01K 67/027; C07H 21/04; C12N 5/06

(52) U.S. Cl. 800/4; 435/354; 536/23.5

(57) ABSTRACT

The invention features a transgenic animal, which expresses a Coxsackie-adenovirus receptor (CAR) in its cells. The invention provides a new animal model for effecting adenovirus-mediated gene delivery and for testing gene function in vivo, and also provides new opportunities to assess gene function in vitro using freshly isolated cells.

### CROSS-REFERENCE TO RELATED APPLICATION

This application claims priority from U.S. Provisional Application Serial No. 60/183,080, filed on Feb. 16, 2000, which is incorporated herein by reference in its entirety.

### STATEMENT AS TO FEDERALLY SPONSORED RESEARCH

This work was funded in part by the National Institutes of Health under Grant No. AI41054. The government has certain rights to this invention.

### FIELD OF THE INVENTION

This invention relates to transgenic animals.

### BACKGROUND OF THE INVENTION

The transfer of genes into animal cells using viral vectors has provided a powerful tool for correcting genetic defects and for studying the cellular processes involving specific genes. Replication defective adenoviral vectors have many advantages as vehicles for gene transfer, e.g., stocks of modified adenoviruses are readily produced in high titer; superinfection with adenoviruses encoding different genes is possible; and gene expression is robust and of relatively long duration (Berkner, K. L. 1988, BioTechniques 6:616-629; Baddada et al., 1995, Curr Top Microbiol Immunol 199, no. Pt 3:297-306; Chartier et al., 1996, J Virol 70, no. 7:4805-10; He et al., 1998, Proc. Natl. Acad. Sci., USA 95:2509-2514). Unlike retrovirus vectors, which require target cells in S phase for vector integration and expression, adenovirus infection and subsequent gene expression can occur in resting cells. Moreover, adenoviruses are primarily maintained in the nuclei of infected cells, in association with the nuclear matrix, without integration (Shenk et al., 1996. Adenoviridas: The viruses and their replication. third ed. In Virology, vol. 1. D. M. K. B. N. Fields, and P. M. Howley, editor. 2 vols. Lippincott-Raven, Philadenoviruselphia, Pa. 2111-2148).

Adenovirus infection requires the presence of the Coxsackie/adenovirus receptor (CAR) for virus attachment to the cell surface and an integrin co-receptor, which promotes virus internalization (Bergelson et al., 1997, Science 275:1320-1323; Bergelson et al., 1998, Journal of Virology 72:415-419; Wickham et al., 1993, Cell 73:309-319; and Nemerow et al., 1999, Microbiology and Molecular Biology Reviews 63:725-734). Both human (h) and murine (m) genes for CAR have been identified and both support infection by human adenovirus when transfected into CAR negative cell lines (Bergelson et al., 1997, supra; Bergelson et al., 1998, supra; Leon et al,. 1998, Proc. Natl. Acad. Sci., USA 95:13159-13164).

### SUMMARY

Many cell types, including lymphocytes, are refractory to adenovirus infection. The present invention is based on the discovery that cells, which are normally refractory to adenovirus infection, can be made susceptible to adenoviruses by introducing a Coxsackie/adenovirus receptor (CAR) gene encoding CAR into the cells. The invention features transgenic animals, which express CAR in at least some of their cells. Thus, cells of the transgenic animals, including cells that are normally refractory to adenovirus infection, e.g., cells of hematopoietic origin, can be made susceptible to adenovirus infection. Based on this discovery, the invention provides a new animal model for effecting adenovirus-mediated gene delivery and for testing gene function in vivo, and also provides new opportunities to assess gene function in vitro using freshly isolated cells.

Accordingly, in one aspect, the invention features a transgenic, non-human animal (e.g., a rodent such as a mouse, or other mammals, such as goats or cows, or birds), where one or more of its cells includes a transgene having a regulatory sequence operably linked to a nucleic acid sequence encoding a Coxsackie/adenovirus receptor (CAR). The transgene is expressed in one or more cells of the transgenic animal resulting in the animal exhibiting increased susceptibility to adenovirus infection as compared to a wildtype animal. The nucleic acid sequence encoding CAR is a nucleic acid sequence substantially identical to a nucleic acid sequence encoding a mouse or human CAR. Any cell, e.g., hematopoietic cells or lymphocytes, can express the transgene. The regulatory sequence can be any regulatory sequence, and the regulatory sequence can be constitutive or inducible. In some embodiments, the regulatory sequence controlling transcription of CAR is different from the regulatory sequence controlling the transcription of an endogenous CAR.

In one embodiment, the transgenic animal can be an animal that develops or has a disorder, e.g., an immunodeficiency disorder such X-linked severe combined immune deficiency, X-linked agammaglobulinemia, or Wiskott-Aldrich syndrome.

In another aspect, the invention features a nucleic acid molecule comprising an MHC class 1 regulatory sequence operably linked to a nucleic acid sequence that encodes a CAR. The DNA fragment can be an integral part of a linear construct or of a vector (e.g., a plasmid or a viral vector).

Also contemplated in the invention is a cell containing the transgene described herein. In one embodiment, the cells can be derived from the transgenic animals of the invention. Examples of such cells include lymphocytes, liver cells, or spleen cells.

The present invention also includes a method of infecting a cell with an adenovirus, by contacting a cell engineered to express a nucleic acid sequence encoding a CAR polypeptide with an adenovirus such that the adenovirus infects the cell. The cell can be a cell that is normally refractory to adenovirus infection, e.g., a hematopoietic cell.

Also included within the scope of the invention is an in vitro method of determining whether an adenoviral vector can infect a cell in vivo. The method includes contacting a cell from the transgenic animal described above in vivo, e.g., a cell that is normally refractory to adenoviral vector infection, with an adenoviral vector comprising a test gene that encodes a test protein; and determining the expression of the test gene in the cell, wherein expression of the test gene in the cell indicates that the cell can be infected with the adenoviral vector.

In another aspect, the invention features a method of testing the efficiency of a regulatory sequence to express a nucleic acid sequence in a cell in vivo. The method includes contacting a cell engineered to express a nucleic acid sequence encoding a CAR polypeptide in vivo with an adenoviral vector comprising a test regulatory sequence operably linked to a reporter gene, and determining the level of expression of the reporter gene in the cell, wherein the level of expression is an indication of the efficiency of the regulatory sequence. The cell can be a tumour cell, e.g., a prostate tumor cell.

A “transgene” is any exogenous nucleic acid sequence that is integrated into the genome of a cell from which a transgenic animal develops and which remains in the genome of the mature animal.

A “transgenic animal” is a non-human animal (e.g., a mammal such as a non-human primate, dog, cat, cow, pig, goat, sheep, horse, rabbit, mouse, rat, guinea pig, or hamster, or a bird such as a chicken) in which one or more of the cells of the animal include a transgene.

A “regulatory sequence” is a DNA sequence that directs the transcription of a gene in a cell.

Expression control or regulatory sequences are “operably linked” to a test nucleic acid when they are positioned to effectively control expression of the test nucleic acid. Typically, the expression control or regulatory sequences are located upstream of the test nucleic acid in terms of the direction of transcription.

A “polypeptide” is any peptide-linked chain of amino acids, regardless of length or post-translational modification. The term “polypeptide” therefore includes peptides and proteins.

A “conservative amino acid substitution” in a polypeptide is one in which an amino acid is replaced with another amino acid having a chemically similar side chain. Families or groups of amino acids having similar side chains have been defined in the art. These families include amino acids with basic side chains (e.g., lysine, arginine, and histidine), acidic side chains (e.g., aspartic acid and glutamic acid), uncharged polar side chains (e.g., glycine, asparagine, glutamine, serine, threonine, tyrosine, and cysteine), nonpolar side chains (e.g., alanine, valine, leucine, isoleucine, proline, phenylalanine, methionine, and tryptophan), beta-branched side chains (e.g., threonine, valine, and isoleucine), and aromatic side chains (e.g., tyrosine, phenylalanine, tryptophan, and histidine). Any member of such groups can be substituted for any other member in the group in a conservative amino acid substitution.

“Percent sequence identity” of two amino acid sequences or of two nucleic acids is determined using the algorithm of Karlin and Altschul (Proc. Natl. Acad. Sci. USA 87:2264-2268, 1990), modified as in Karlin and Altschul (Proc. Natl. Acad. Sci. USA 90:5873-5877, 1993). Such an algorithm is incorporated into the NBLAST and XBLAST programs of Altschul et al. (J. Mol. Biol. 215:403-410, 1990). BLAST nucleotide searches are performed with the NBLAST program, score=100, wordlength=12 to obtain nucleotide sequences homologous to a nucleic acid molecules of the invention. BLAST protein searches are performed with the XBLAST program, score=50, wordlength=3 to obtain amino acid sequences homologous to a test polypeptide. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., XBLAST and NBLAST) are used. See http://www.ncbi.nlm.nih.gov.

“Moderate hybridization conditions” are defined as equivalent to hybridization in 2×sodium chloride/sodium citrate (SSC) at 30° C., followed by one or more washes in 1×SSC, 0.1% SDS at 50-60° C. “Highly stringent conditions” are defined as equivalent to hybridization in 6×sodium chloride/sodium citrate (SSC) at 45° C., followed by one or more washes in 0.2×SSC, 0.1% SDS at 50-65° C.

By “substantially identical” is meant a nucleic acid sequence that shares at least 70% sequence identity with another sequence, e.g., a sequence that encodes CAR, or a sequence that hybridizes under conditions of moderate stringency to another sequence, e.g., a sequence that encodes CAR. Of course, a higher percentage of sequence identity can be used such as 80, 85, 90, 95, or even higher percentages.

An “isolated nucleic acid” is a nucleic acid free of the genes that flank the nucleic acid of interest in the genome of the organism or virus in which the gene of interest naturally occurs. The term therefore includes a recombinant DNA incorporated into an autonomously replicating plasmid. It also includes a separate molecule such as a cDNA, a genomic fragment, a fragment produced by polymerase chain reaction, or a restriction fragment. It also includes a recombinant nucleotide sequence that is part of a hybrid gene, i.e., a gene encoding a fusion protein. An isolated nucleic acid is substantially free of other cellular or viral material (e.g., free from the protein components of a viral vector), or culture medium when produced by recombinant techniques, or substantially free of chemical precursors or other chemicals when chemically synthesized.

By “disorder” is meant any disturbance to the regular or normal functions of an animal.

A “primary cell” is a cell taken directly from an animal and, which is not immortalized.

Unless otherwise defined, all technical and scientific terms used herein have the same meaning as commonly understood by one of ordinary skill in the art to which this invention belongs. Although suitable methods and materials for the practice or testing of the present invention are described below, other methods and materials similar or equivalent to those described herein, which are well known in the art, can also be used. All publications, patent applications, patents, and other references mentioned herein are incorporated by reference in their entirety. In case of conflict, the present specification, including definitions, will control. In addition, the materials, methods, and examples are illustrative only and not intended to be limiting.

Various advantages arise from the present invention, which provides transgenic, non-human animals that express in one or more of their cells an exogenous Coxsackie/adenovirus receptor. For example, cells of the transgenic animals are susceptible or have enhanced susceptibility to adenovirus infection compared to similar cells of wild-type animals. The transgenic animals have many uses. For example, they can serve as excellent models for gene replacement therapy, and can be used to study gene function in a wide variety of cells, particularly cells that are normally refractory to adenovirus infection, such as lymphocytes.

Other features and advantages of the invention will be apparent from the following detailed description, and from the claims.

### BRIEF DESCRIPTION OF THE DRAWINGS

FIG. 1 is a schematic drawing showing the H-2K-human-CAR construct used to generate the transgenic mice of the invention. Locations of PCR primer sets used to positively identify transgene founders are also indicated.

FIGS. 2A to 2D are a series of photographs showing RT-PCR analysis of the spleen (2A), thymus (2B), testis (2C), and kidney (2D) from B6 (c) and hCAR founders, 12, 24, 28, and 31. Transcripts were detected using the internal hCAR primers. RT-PCR for β actin was used as control (shown in lower view of each figure).

FIGS. 3A-D are four flow cytometric graphs showing surface expression of hCAR on founder line 28 cells. Spleen (FIG. 3A), lymph node (FIG. 3B), thymus (FIG. 3C) and bone marrow cells (FIG. 3D) stained with: control (^{•}), or anti-hCAR(−) ascites

FIG. 4 is an autoradiograph showing the detection of hCAR protein on transgenic splenic B and T cells of one founder by immunoprecipitation of biotinylated surface proteins with RcmB.

FIGS. 5A-B are two flow cytometric graphs showing the surface expression of αᵥ integrin on B6 and hCAR⁺ spleen cells: unstained cells (^{•}), cells stained with anti-αᵥ-PE (−) or Ig-PE (−−) control antibody.

FIGS. 5C-D are two flow cytometric graphs showing the surface expression of β₃ integrin on B6 and hCAR⁺ spleen cells: unstained cells (^{•′•}), cells stained with anti-β₃-PE (−) or Ig-PE (−−) control antibody.

FIGS. 6A-J are a series of flow cytometric graphs showing infection of transgene lymphoid cell populations with adenovirus encoding a Green Fluorescent Protein (GFP) reporter gene. Cells were prepared from B6 and a transgenic human CAR (hCARtg) founder line. All populations shown were gated on lymphocytes: FIGS. 6A-B are graphs showing B220⁺ spleen cells stimulated with anti-Ig+IL-4 from B6 mice and transgenic human CAR mice, respectively; FIGS. 6C-D are graphs showing unstimulated B220⁺ bone marrow cells from B6 mice and transgenic human CAR mice, respectively; FIGS. 6E-F are graphs showing Thy 1.2⁺ lymph node cells stimulated with anti-CD3+IL-2 from B6 mice and transgenic human CAR mice, respectively; FIGS. 6G-H are graphs showing Thy 1.2⁺ thymocytes stimulated with ConA+IL-2 from B6 mice and transgenic human CAR mice, respectively; and FIGS. 6I-J are graphs showing unstimulated B220⁺ peritoneal B1a cells (gated on CD5⁺ and Mac-1^{lo}) from B6 mice and transgenic human CAR mice, respectively.

FIGS. 7A-D are four flow cytometric graphs showing the infection of non-lymphoid peritoneal and bone marrow populations from B6 and hCAR transgenic mice with adenovirus CMV(cytomegalovirus)-GFP. Bone marrow cells, B220 negative (FIGS. 7A-B), and peritoneal cells, Thy 1.2 (FIGS. 7C-D), are gated for non-lymphoid cells by forward and side scatter.

FIGS. 8A-J are flow cytometric graphs showing promoter and activator dependent expression of adenovirus encoded GFP. B6 and transgenic splenic B cells were exposed to either adenovirus CMV-GFP or adenovirus EF1-GFP that encode a GFP reporter gene under control of an hCMV or hEF1α promoter, respectively. FIGS. 8A-D show transgenic splenic B cells exposed to adenovirus CMV-GFP. Untreated cells are shown in FIG. 8A, and cells treated with anti-Ig+IL-4, LPS, and PMA+Ionomycin are shown in FIGS. 8B-D, respectively. FIGS. 8E-H show transgenic splenic B cells exposed to adenovirus CMV-GFP. Untreated cells are shown in FIG. 8E, and cells treated with anti-Ig+IL-4, LPS, and PMA+Ionomycin are shown in FIGS. 8F-H, respectively. FIGS. 8I-J show splenic B cells from B6 mice exposed to adenovirus CMV-GFP. FIG. 8I shows untreated cells and FIG. 8J shows cells that have been treated with anti-Ig+IL-4.

FIGS. 9A-L are flow cytometric graphs showing promoter and activator dependent expression of adenovirus encoded Bex, which is a modified form of GFP. B6 and transgenic splenic B cells were exposed to either adenovirus AdCMV-Bex or adenovirus AdEF1-GFP that encode a Bex or GFP reporter gene under control of an hCMV or hEF1α promoter, respectively. FIGS. 9A-D show transgenic splenic B cells exposed to AdCMV-Bex. Untreated cells are shown in FIG. 9A, and cells treated with anti-Ig+IL-4, LPS, and PMA+Ionomycin are shown in FIGS. 9B-D, respectively. FIGS. 9E-H show transgenic splenic B cells exposed to AdEF1-GFP. Untreated cells are shown in FIG. 9E, and cells treated with anti-Ig+IL-4, LPS, and PMA+Ionomycin are shown in FIGS. 9F-H, respectively. FIGS. 9I-J show splenic B cells from B6 mice exposed to AdCMV-Bex. FIG. 9I shows untreated cells and FIG. 9J shows cells which have been treated with anti-Ig+IL-4. FIGS. 9K and 9L show splenic B cells from B6 mice exposed to AdEF1-GFP. FIG. 9K shows untreated cells and FIG. 9L shows cells that have been treated with anti-Ig+IL-4.

### DETAILED DESCRIPTION

The transgenic, non-human animals of the invention express in one or more of their cells an exogenous coxsackie/adenovirus receptor (CAR). Expression of transgenic CAR in a wild-type cell that normally expresses CAR results in a cell that has enhanced susceptibility to adenovirus infection compared to the wild-type cell. Expression of transgenic CAR in a wild-type cell that normally does not express CAR results in a cell that expresses the receptor and is no longer refractory to adenovirus infection. The new genetically modified animals have many uses. For example, these animals are excellent models for gene replacement therapy, and can be used for studies of gene function in a wide variety of cells, particularly in those cells that are normally refractory to adenovirus infection such as lymphocytes.

### Methods of Making Transgenic Animals That Express CAR

### Transgene

The transgene includes a CAR nucleic acid sequence operably linked to a regulatory sequence, which is introduced into both the somatic and germ cells, or only some of the somatic cells of an animal. The transgene is introduced in such a manner that the inserted transgene can be expressed and produced in the animal. The CAR nucleic acid sequence can be a CAR gene, a portion of the gene, a nucleic acid sequence that hybridizes under moderate or high stringency conditions to a known CAR sequence and encodes a polypeptide which has a CAR function, or a sequence that is substantially identical to a known CAR sequence. CAR genes for various species have been reported. Examples include human CAR (Bergelson, 1997, supra (Genbank Accession #Y07593)), and mouse CAR (Genbank Accession #Y11929).

### Regulatory Sequences

The expression of CAR is driven by a regulatory sequence. The term regulatory sequence includes promoters, enhancers and other expression control elements. It will be appreciated that the appropriate regulatory sequence depends on such factors as the future use of the transgenic animal, and the level of expression of the test polypeptide desired. A person skilled in the art would be able to choose the appropriate regulatory sequence. For example, the transgenic animals described herein can be used to determine the role of a test polypeptide in a particular cell type, e.g., a hematopoietic cell. In this case, a regulatory sequence that drives expression of the transgene ubiquitously, or a hematopoietic-specific regulatory sequence that expresses the transgene only in hematopoietic cells, can be used. Expression of CAR in the hematopoietic cell means that the cell is now susceptible to infection by an adenovirus encoding the test protein. In another example, a regulatory sequence that expresses the transgene during a disorder is chosen. In this example, the transgenic animal can be used to determine the role of a test polypeptide in a particular disorder. Examples of various regulatory sequences are described below.

The regulatory sequence can be the same as the endogenous regulatory sequence, or different. It can be inducible or constitutive. Suitable constitutive regulatory sequences include the regulatory sequence of a housekeeping gene such as the α-actin regulatory sequence, or may be of viral origin such as regulatory sequences derived from mouse mammary tumor virus (MMTV) or cytomegalovirus (CMV).

Alternatively, the regulatory sequence can direct transgene expression in specific organs or cell types. Several tissue-specific regulatory sequences are known in the art including the albumin regulatory sequence for liver (Pinkert et al., 1987, Genes Dev. 1:268-276); the endothelin regulatory sequence for endothelial cells (Lee, 1990, J. Biol. Chem., 265:10446-50); the keratin regulatory sequence for epidermis; the myosin light chain-2 regulatory sequence for heart (Lee et al., 1992, J. Biol. Chem., 267:15875-85), and the insulin regulatory sequence for pancreas (Bucchini et al., 1986, Proc. Natl. Acad. Sci., 83:2511-2515), or the vav regulatory sequence for hematopoietic cells (Oligvy et al., Proc. Natl. Acad. Sci., U S A., 1999, 96:14943-14948). Another suitable regulatory sequence, that directs constitutive expression of transgenes in cells of hematopoietic origin, is the murine MHC class I regulatory sequence (Morello et al., 1986 , EMBO J. 5:1877-1882). Since MHC expression is induced by cytokines, expression of a test gene operably linked to this regulatory sequence can be upregulated in the presence of cytokines.

In addition, expression of the transgene can be precisely regulated, for example, an inducible regulatory sequence such as a regulatory sequence that is sensitive to certain physiological regulators, e.g., circulating glucose levels, or hormones (Docherty et al., 1994 FASEB J. 8:20-24) can be used.

### Procedures for Making Transgenic, Non-Human Animals

A number of methods have been used to obtain transgenic, non-human animals, which are animals that have gained an additional gene by the introduction of a transgene into their cells (e.g., both the somatic and germ cells), or into an ancestor's germ line.

Methods for generating transgenic animals include introducing the transgene into the germ line of the animal. One method is by microinjection of a gene construct into the pronucleus of an early stage embryo (e.g., before the four-cell stage; Wagner et al., 1981, Proc. Natl. Acad. Sci., USA, 78:5016; Brinster et al., 1985, Proc. Natl. Acad. Sci., USA, 82:4438). Alternatively, the transgene can be introduced into the pronucleus by retroviral infection. A detailed procedure for producing such transgenic mice has been described (see e.g., Hogan et al., Manipulating the Mouse Embryo, Cold Spring Harbour Laboratory, Cold Spring Harbour, N.Y. (1986); U.S. Pat. No. 5,175,383 (1992)). This procedure has also been adapted for other animal species (e.g., Hammer et al., Nature 315:680 (1985); Murray et al., Reprod. Fert. Devl. 1:147 (1989); Pursel et al., Vet. Immunol. Histopath. 17:303 (1987); Rexroad et al., J. Reprod. Fert. 41 (suppl):119 (1990); Rexroad et al., Molec. Reprod. Devl. 1:164 (1989); Simons et al., BioTechnology 6:179 (1988); Vize et al., J. Cell. Sci. 90:295 (1988); and Wagner, J. Cell. Biochem. 13B (suppl):164 (1989)).

In brief, the procedure involves introducing the transgene into an animal by microinjecting the construct into the pronuclei of the fertilized mammalian egg(s) to cause one or more copies of the transgene to be retained in the cells of the developing mammal(s). Following introduction of the transgene construct into the fertilized egg, the egg may be incubated in vitro for varying amounts of time, or reimplanted a surrogate host, or both. One common method is to incubate the embryos in vitro for about 1-7 days, depending on the species, and then reimplant them into the surrogate host. The presence of the transgene in the progeny of the transgenically manipulated embryos can be tested by Southern blot analysis of a segment of tissue.

Another method for producing germ-line transgenic animals is through the use of embryonic stem (ES) cells. The gene construct can be introduced into embryonic stem cells by homologous recombination (Thomas et al., Cell, 51:503 (1987); Capecchi, Science, 244:1288 (1989); Joyner et al., Nature, 338:153 (1989)) in a transcriptionally active region of the genome. A suitable construct can also be introduced into embryonic stem cells by DNA-mediated transfection, such as by electroporation (Ausubel et al., Current Protocols in Molecular Biology, John Wiley & Sons (1987)). Detailed procedures for culturing embryonic stem cells (e.g., ES-D3, ATCC# CCL-1934, ES-E14TG2a, ATCC# CCL-1821, American Type Culture Collection, Rockville, Md.) and methods of making transgenic animals from embryonic stem cells can be found in Teratocarcinomas and Embryonic Stem Cells, A Practical Approach, ed. E. J. Robertson (IRL Press, 1987). In brief, the ES cells are obtained from pre-implantation embryos cultured in vitro (Evans, M. J., et al., 1981, Nature, 292:154-156). Transgenes can be efficiently introduced into ES cells by DNA transfection or by retrovirus-mediated transduction. The resulting transformed ES cells can thereafter be combined with blastocysts from a non-human animal. The ES cells colonize the embryo and contribute to the germ line of the resulting chimeric animal.

In the above methods, the transgenic construct can be introduced as a linear construct, a circular plasmid, or a viral vector, which can be incorporated and inherited as a transgene integrated into the host genome. The transgene can also be constructed so as to permit it to be inherited as an extrachromosomal plasmid (Gassmann, M. et al., 1995, Proc. Natl. Acad. Sci. USA 92:1292). A plasmid is a DNA molecule that can replicate autonomously in a host.

The transgenic, non-human animals can also be obtained by infecting new cells either in vivo (e.g., direct injection), ex vivo (e.g., infecting the cells outside the host and later reimplanting), or in vitro (e.g., infecting the cells outside host) with a recombinant viral vector carrying the CAR gene. Examples of suitable viral vectors include recombinant retroviral vectors (Valerio et al., 1989, Gene, 84:419; Scharfman et al., 1991, Proc. Natl. Acad. Sci., USA, 88:462; Miller, D. G. & Buttimore, C., 1986, Mol. Cell. Biol., 6:2895), recombinant adenoviral vectors (Freidman et al., 1986, Mol. Cell. Biol., 6:3791; Levrero et al., 1991, Gene, 101:195), and recombinant Herpes simplex viral vectors (Fink et al., 1992, Human Gene Therapy, 3:11). Recombinant retroviral vectors capable of transducing and expressing structural genes (e.g., CAR genes) inserted into the genome of a cell are produced by transfecting the recombinant retroviral genome into suitable packaging cell lines such as PA317 and Psi-CRIP (Cornette et al., 1991, Human Gene Therapy 2:5-10; Cone et al., 1984, Proc. Natl. Acad. Sci., USA, 81:6349). Recombinant adenoviral vectors can be used to infect a wide variety of cells and tissues in susceptible hosts (e.g., rat, hamster, dog, and chimpanzee) (Hsu et al., 1992, J. Infectious Disease, 166:769), and also have the distinct advantage of not requiring mitotically active cells for infection.

Clones of the non-human transgenic animals described herein can be produced according to the methods described in Wilmut et al. ((1997) Nature, 385:810-813) and PCT publication Nos. WO 97/07668 and WO 97/07669. In brief, a cell, e.g., a somatic cell from the transgenic animal, can be isolated and induced to exit the growth cycle and enter the Gₒ phase to become quiescent. The quiescent cell can then be fused, e.g., through the use of electrical pulses, to an enucleated oocyte from an animal of the same species from which the quiescent cell is isolated. The reconstructed oocyte is then cultured such that it develops into a morula or blastocyte and is then transferred to a pseudopregnant female foster animal. Offspring borne of this female foster animal will be clones of the animal from which the cell, e.g., the somatic cell, was isolated.

Once the transgenic animal is produced, cells of the transgenic animal and cells from a control animal are screened to determine for the presence of a CAR nucleic acid sequence, e.g., using polymerase chain reaction (PCR). Alternatively, the cells can be screened to determine if CAR mRNA is expressed (e.g., CAR mRNA can be detected by standard procedures such as Northern blot analysis or reverse transcriptase-polymerase chain reaction (RT-PCR); Sambrook et al., Molecular Cloning—A Laboratory Manual, (Cold Spring Harbor Laboratory, 1989)) or if CAR protein is produced (e.g., CAR can be detected using Western blot analysis; Sambrook et al., Molecular Cloning—A Laboratory Manual, (Cold Spring Harbor Laboratory, 1989)).

The transgenic animals of the present invention can be homozygous or heterozygous and both can support adenovirus infection.

### Adenoviral Vectors

The new transgenic animals express CAR in a large variety of different cells. Cells expressing CAR are susceptible to adenovirus infection, thus allowing the transfer of a test nucleic acid sequence (e.g., a test gene) into the cells using an adenoviral vector. To transfer a test nucleic acid sequence into a cell using an adenoviral vector, the nucleic acid is first inserted into the vector.

A number of adenoviral vectors have been developed for the transfer of genes into cells (Berkner et al., 1988, BioTechniques, 6:616629). Constitutive high-level expression of the transferred nucleic acid sequence has been achieved. These vectors have the inherent advantage over retroviral vectors in not requiring replicating cells for infection, making them suitable vectors for somatic gene therapy (Mulligan, R. C., Science, 260:926-932 (1993)).

Strategies for generating replication defective adenoviral recombinants have been described (Berkner et al., 1988, BioTechniques, 6:616-629). For example, plasmid pMLP6 (Logan et al., Proc. Natl. Acad. Sci. USA 81:3655-3659 (1984) carries the adenovirus 5 genome with the E1 region deleted. An expression cassette containing a regulatory sequence operably linked to a test nucleic acid sequence of test gene can be cloned into the adenovirus 5 plasmid. The entire recombinant adenovirus genome is then generated by mixing the linearized adenovirus plasmid with a subgenomic fragment of adenovirus DNA representing 3.85-100 map units (Berkner et al., BioTechniques 6:616-629 (1988)). The DNAs are then introduced into a cell line such as the kidney 293-cell line. Intermolecular recombination across appropriate segments of the plasmid and the subgenomic fragment of adenoviral DNA will result in the production of replication defective recombinant adenoviral genomes carrying the test nucleic acid sequence or test gene. The recombinant genomes will emerge from the 293 cell lines as packaged viral particles shed into the medium. Modifications of this design have resulted in high-level expression vectors (Berkner et al., 1988, BioTechniques 6:616-629) by incorporating regions of the so-called major late promoter and the tripartite leader elements in the vector (Berkner et al., Nuc. Acid Res. 11:6003-6020 (1983); and Logan et al., Proc. Natl. Acad. Sci. USA 81:3655-3659 (1984).

The adenoviral vector can be delivered to a transgenic animal described herein, for example, by oral ingestion, intravenous injection, intramuscular injection, local administration, gastric intubation or broncho-nasal spraying. Alternatively, cells can be isolated from the transgenic animal, infected with the adenovirus vector in vitro, e.g., by transfection, and returned to transgenic or non-transgenic recipients. Hematopoietic stem cells, peritoneal lymphocytes, or macrophages, peripheral blood lymphocytes, and other cell types are suitable for this purpose.

### Use of Transgenic Animals that Express CAR

The new transgenic animals enable persons skilled in the art to transfer test nucleic acid sequences (e.g., genes) into a variety of cells by in vivo infection. Importantly, a replication defective recombinant adenoviral vector containing a test nucleic acid sequence can infect cells that are normally refractory, or only mildly susceptible, to adenovirus infection. An example of such a cell is a lymphocyte.

The new transgenic animals have many uses as described below.

### Delivery of Test Nucleotide Sequences in Vivo

The transgenic animals of the present invention provide a new way to deliver test nucleic acid sequences to cells in vivo. This can be accomplished by administering to one of the new transgenic animals a recombinant adenoviral vector containing a regulatory sequence operably linked to a test nucleic acid. The test nucleic acid sequence can be DNA or RNA, and can be synthetic, e.g., a gene fusion, or natural. The test sequence can encode a great variety of polypeptides including hormones, cytokines, antigens, antibodies, enzymes, clotting factors, transport proteins, receptors, regulatory proteins, or structural proteins, trancription factors, or encoded anti-sense sequences capable of specifically inhibiting the production of endogenous polypeptides. The regulatory sequence can direct continuous expression of the test nucleic acid sequence in a variety of cell types, or can direct constitutive tissue-specific or cell-type specific expression of the test nucleic acid sequence.

The new transgenic animals can be used to examine the role of one or more test nucleic acid sequences in a cell. For example, a protein suspected of being an inhibitor or an activator of a cellular process (e.g., differentiation, receptor signaling, apoptosis, cell growth, import or export of toxins and nutrients) can be introduced into a cell and its ability to inhibit or activate the cellular process assessed.

In one example, a recombinant adenoviral vector containing a gene of interest operably linked to a regulatory sequence is delivered to a cell. The gene of interest is overexpressed in the cell (optionally an inducing agent can also be administered to increase the level of expression of the gene of interest by acting on the regulatory sequence, e.g., cytokines can be used to upregulate MHC class 1 promoter activity) and the role of the encoded gene product determined. In particular, the transgenic animals described herein can be used to evaluate the role of particular gene products in cells of the hematopoietic system. For example, replication defective recombinant adenoviral vector containing a regulatory sequence, e.g., of the vav gene, operably linked to a gene of interest, e.g., bcl-2, Bruton's tyrosine kinase, interleukin common gamma chain, multi-drug resistance proteins (MDR), Fas, Fas ligand, telomoerase, p53, p16^{INKa4}, and constitutively active Rb. The vav gene is active throughout the hemapoietic system, but rarely outside it, and its regulatory sequences can be used to specifically express a protein of interest in cells of the hematopoietic system.

In another example, subtractive cloning schemes can be used to identify genes whose products regulate a cellular process such as apoptosis. To further characterize the role of such a gene, the gene is inserted into a replication defective recombinant adenoviral vector and the vector is administered to a transgenic animal described herein. The role (e.g., the apoptotic role) of the protein in a cell can then be assessed.

The invention further provides a means of investigating the role of a gene of interest (e.g., a cancerous gene, an apoptotic gene, a gene which encodes a signaling molecule, a regulator of cell cycle progression, toxin exporters, or genes that foster cell transformation e.g. by maintaining telomerase function) by using an adenoviral vector to deliver an antisense nucleic acid molecule or a ribozyme to a cell. In one example, adenoviruses can be used to deliver an antisense DNA molecule. The antisense DNA binds to the coding strand of the gene of interest (e.g., MDR1, telomerase, Rb, virally encoded enzymes, transcription factors) inhibiting translation. Alternatively, the gene of interest, or a portion thereof, is cloned into the adenoviral vector in such a way that it is operably linked to a regulatory sequence in an antisense orientation and administered to the transgenic animal of the invention. Expression of the gene of interest results in an RNA molecule that is antisense to the gene of interest inhibiting transcription of the gene. Since expression of the gene of interest is inhibited, its cellular role can be determined.

### Delivery of a Test Nucleotide Sequence in Vitro

The new transgenic, non-human animals can also be used as a source of cells, tissues, or organs. Cells, tissues, and organs can be cultured using standard tissue culture techniques (e.g., A Dissection and Tissue Culture Manual of the Nervous System, Shahar et al. (eds), Alan R. Liss Inc. (1989); Animal Cell Culture, Pollard et al. (eds), Humana Press (1989)).

For example, primary cells, e.g., lymphocytes, can be isolated from a transgenic animal and grown in the appropriate media. The cells are infected with a replication deficient recombinant adenoviral vector containing a regulatory sequence operably linked to a test nucleic acid sequence. Optionally, cells can be activated, e.g., by exposing cells to phorbol 12-myristate 13-acetate (PMA), phytohemagglutinin (PHA), concanavalin A (Con A), or agarose-bound phytohemagglutinin, lipopolysaccharide, interferon-alpha, interleukin-2 or chemotherapeutic drugs such as Paclitaxel. Activating lymphocytes may result in higher levels of expression of the test nucleic acid sequence, became transcription factors, which are necessary for activation of the promoter may not be available in resting cells. Expression of the transfected test nucleic acid sequence can be examined by Northern blot analysis (Sambrook et al., Molecular Cloning—A Laboratory Manual, (Cold Spring Harbor Laboratory, 1989).

The primary cells derived from the transgenic animals described herein can be used to study the function of a test gene. These cells can be used, for example, to determine a binding partner for a test polypeptide. For example, the binding partner of a T-cell receptor signaling molecule can be determined by infecting lymphocytes derived from the transgenic animal with a recombinant adenoviral vector that encodes a test polypeptide. The lymphocytes are then activated, lysed and the test polypeptide isolated, e.g., by immunoprecipitation. The presence of a binding partner for the test polypeptide can be analyzed by Western blot analysis.

### Delivery of a Test Nucleotide Sequence Ex Vivo

Primary cells isolated from an animal can be genetically modified and implanted back into the animal. These engineered cells can serve, for example, as surrogate tissues or organs (Mendell, J. R. et al., N. Engl. J. Med., 333:832-838 (1995), or as neo-organs (Thompson et al., Proc. Natl. Acad. Sci., USA, 86:7928-7932 (1989). In one example, lymphocytes that are normally refractory to adenovirus infection can be isolated from the transgenic animal described herein and transduced with a replication defective recombinant adenoviral vector carrying an expression cassette including a test nucleic acid sequence under the transcriptional control of an appropriate regulatory sequence. The genetically modified lymphocytes can then be implanted back into the animal. Alternatively, lymphocytes from a wild-type animal can be isolated and transfected with a vector that contains CAR operably linked to an appropriate regulatory sequence. The lymphocytes can be implanted back into the wild-type animal and these lymphocytes can be infected with a replication defective recombinant adenoviral vector containing a test nucleic acid sequence.

In another example, myoblasts can be isolated from muscle biopsies of transgenic animals such as mice. The myoblasts can then be infected with adenoviruses such that they express high levels of a test protein. The test protein expressing myoblasts can then be transferred into muscle by direct injection of the cells. Previous experience with murine myoblast has demonstrated that the injected myoblasts will fuse into pre-existing multinucleate myofibrils (Dhawan et al., Science, 254:1509-1512 (1991); and Barr et al., Science, 254:1507-1509 (1991)). The differentiated muscle fibers will maintain a high level of expression of the transgene (Yao et al., Proc. Natl. Acad. Sci., USA, 89:3357-3361 (1992)).

### Methods of Testing Regulatory Sequences

Many differences exist in regulated gene expression between primary cells and cultured cell lines. The long standing observation that the human cytomegalovirus (CMV) promoter can successfully direct gene expression in many transformed murine lymphocyte lines but not primary lymphocytes, is an example of this point. Because the efficiency of promoter driven expression is dependent on the availability of specific transcription factors, promoter choice is a critical variable in the development of vectors for gene transfer studies or therapy. The CAR transgenic mouse described herein affords the opportunity to analyze whether certain regulatory sequences function in particular cell types in vivo and whether these regulatory sequences can provide sustained expression of the transgene in a cell of interest in vivo.

For example, the ability of a regulatory sequence (e.g., a hybrid regulatory sequence made up of regulatory sequence elements from a number of different genes or which is synthetic) to control expression of a nucleic acid in a cell in vivo can be tested as follows. A replication defective recombinant adenoviral vector comprising a test regulatory sequence operably linked to a reporter gene is administered to a transgenic animal described herein. Expression of the reporter gene in the cell of interest is determined, e.g., by Northern blot analysis. The level of expression is an indication of the expression efficiency of the promoter. The reporter gene can be any gene that functions as a reporter. Examples of reporter genes include B-lactamase, chloramphenicol acetyltransferase (CAT), adenosine deaminase, aminoglycoside phosphotransferase, dihydrofolate reductase, lacZ, and xanthine guanine phosphoribosyltransferase.

### Animal Models for Diseases

Gene therapy is of potential use in the treatment of many disorders including cancer, diabetes, acquired immunodeficiency syndrome, rheumatoid arthritis, metabolic abnormalities, or sickle cell anemia. A key to gene therapy is the specific delivery and expression of test genes in target cells. Presently, gene therapy is mostly performed in non-human animal models that have disorders similar to known human disorders. The invention further features manipulating these animals to express CAR in their tissues and cells, which provides disease models in which one or more cells that were previously refractory to gene delivery by adenoviruses are now susceptible to adenovirus infection. These mice are useful for in vivo testing of gene replacement therapy under conditions that closely approximate therapeutic regimens.

### Cancer

A variety of genetic abnormalities arise in cancers that contribute to neoplastic transformation and malignancy. Instability of the genome generates mutations that alter cell proliferation, angiogenesis, metastasis, and tumor immunogenicity. Although there is a better understanding of the molecular basis of cancer, many of the malignancies remain resistant to treatment.

The present invention provides a method of studying cancer in vivo. Using a recombinant adenoviral vector, a test nucleic acid sequence-encoding CAR can be delivered to a cancerous cell allowing the study of a tumor, e.g., tumor initiation and progression to malignancy in vivo. The direct transmission of a recombinant test gene into an established tumor in vivo can be used to determine whether the test gene is useful for treating the tumor. The transgenic animal described herein can be used directly, e.g., a virus or drug can be administered to a CAR transgenic animal to induce neoplasia, or oncogenic transgenes can be introduced into the Car transgenic animal. A large number of different cancers can be studied such as prostate cancer, colon cancer, renal cell cancer, breast adenocarcinoma, hepatoma, lung cancer and pancreatic cancer, leukemia, lymphomas, melenoma, and multiple myeloma.

In one example, a test nucleic acid sequence is delivered to a tumor. The test sequence encodes a protein that is suspected of having the ability to monitor the traffic of lymphocytes into the tumor. Alternatively, the test nucleic acid sequence encodes a cytokine or co-stimulatory molecule in a tumor cell. The ability of the encoded cytokine to stimulate the host's immune response against the tumor can be tested. The transgenic animals described herein are discussed below with relevance to prostate cancer.

### Prostrate Cancer

Prostate cancer is the most common malignancy in males and is the second most common cause of cancer mortality in American men. Polymorphisms have been identified in two genes, the 17-hydroxylase cyctochrom P450 gene (CYP17) and the steroid 5-reductase type II gene (SRD5A2). These genes are involved with androgen biosynthesis and metabolism.

Animal models for prostate cancer have been established. For example, Greenberg (P.N.A.S., 1995, 92:3439-3443) generated transgenic mice which develop prostate cancer by introducing into the germ line of the mice a construct comprised of the minimal −426/+28 rat probasin regulatory sequence operably linked to the simian virus 40 large tumor antigen-coding region. High-level expression of this gene in prostate cells caused metastatic prostate cancer in the transgenic mice.

To study the efficacy of a test nucleic acid sequence in the treatment of prostate cancer, the present invention encompasses a non-human animal that has prostate cancer and expresses CAR, e.g., human CAR, in a prostate tumor cell. Such non-human animals can be generated in many ways including crossbreeding a transgenic non-human animal that expresses a gene that causes prostate cancer (simian virus 40 large tumor antigen-coding region) and a transgenic non-human animal that overexpresses CAR. In another example, the CAR transgene described herein can be integrated into a germline of a non-human animal that has prostate cancer. In yet another example, a transgene that causes prostate cancer and the transgene of the invention are both integrated into the germline of a non-human animal. In yet another example, a transgenic non-human animal that expresses and encodes CAR can be manipulated such that it develops prostate cancer. In still another example, a plasmid containing a CAR sequence is operably linked to a regulatory sequence (e.g., a prostate specific regulatory sequence) and is introduced into a primary prostate tumor sample isolated from a human patient. The resulting tumor cells that express CAR are then transplanted into an athymic nude animal.

To determine the efficacy of a particular gene product to treat prostate cancer, a replication deficient recombinant adenoviral vector expressing a test gene under the control of a regulatory sequence is administered to the animal described above and the effect (if any) of the gene product on tumor viability is assessed. Typically, the regulatory sequence is a prostate tissue-specific regulatory sequence. Many prostate tissue-specific regulatory sequences are known in the art including probasin, mouse mammary tumor virus long-terminal repeat (Steiner et al., Cancer Gene Ther., 199, 6:456-464), prostate-specific transglutaminase gene (TGM4), or PSA enhancer sequence (Latham et al. Cancer Res, 2000, 60:334-41). Alternatively, a prostate tumor-specific regulatory sequence, or a regulatory sequence from a gene whose expression is upregulated in prostate tumor cells can be used, e.g., prostate-specific antigen (PSA) or pseudoautosomal gene MIC2.

The test nucleic acid sequence introduced into the cancerous prostate cells can be any nucleotide sequence, e.g., a natural sequence or a synthetic sequence. Examples of test nucleic acid sequences include proapoptotic gene sequences such as BAX (Tai et al., Cancer Res, 1999, 59:2121-6), bcl-2, or FAS or bcl-2 ribozyme. Alternatively, the test nucleotide sequence can encode a tumor suppressor gene (e.g., p53, p21, p16, Rb such as a constitutively active Rb with deleted phosphorylation sites; Knudsen et. al., Oncogene, 1999, 18:5239-45), promyelcytic leukemia gene or a polypeptide that is toxic to the cancerous tumor cells (e.g., osteocalcin-thymidine kinase).

The present invention can also be used to test a regulatory sequence for its ability to drive efficient expression of a test nucleic acid sequence in a prostate tumor cell in vivo. For example, the expression efficiency of a regulatory sequence can be tested in vivo by delivering an adenovirus vector comprising a test regulatory sequence operably linked to a reporter gene to a prostate tumor cell. The level of expression of the reporter in the tumor cell is an indication of the expression efficiency of the promoter. Examples of regulatory sequences that can be used include probasin, MMLV LTR, short PSA, EF1a, CMV, with and without the PSA enhancer. Examples of reporter genes that can be used include LacZ reporter and beta-gal staining of tissue sections

### Primary Immunodeficiency Disorders

Primary immunodeficiency disorders encompass a heterogeneous group of inherited diseases that have in common increased susceptibility to infection. The disorders include those that cause defects in essential cellular elements that result in the dysfunction of multiple types of immune cells (e.g., severe combined immunodeficiency (SCID), defects that primarily effect one type of cell involved in immune function (e.g., X-linked agammaglobulinemia (XLA), or defects that affect a wide variety of cells that include cells that are involved in immune function (e.g., Wiskott-Aldrich syndrome).

Mice that are naturally immunodeficient, or transgenic mice that were generated to have an immunodeficiency disease, can be further manipulated to have increased susceptibility to adenovirus infection by introducing CAR into the tissues and cells of the animal. This may be accomplished by combining the genotypes of different mice by crossbreeding the animals. Alternatively, a transgene, which expresses the CAR receptor, can be integrated into the germline of a non-human animal, which has an immunodeficiency disorder. In another example, a transgene that causes an immunodeficiency disorder and the transgene of the present invention can be inserted into a germline of a non-human animal. Accordingly, these animals provide a good gene therapy model for immunodeficiency disorders. An illustrative example is given below.

X-linked SCID syndrome is caused by a mutation in the gamma chain of the IL-2 receptor (denoted common gamma chain). A transgenic mouse can be generated by integrating into the germline of an animal a transgene that causes SCID (e.g., a mutation in the gamma chain of the IL-2 receptor), and a transgene of the present invention. The transgenic animal can be used to determine if a test nucleic acid sequence can correct or treat the disease.

Likewise, XLA can be modeled in CBA/N mice which have a mutation in Bruton's tyrosine kinase (BTK) (Rawlings et. al., 1993, Science 261:358-61). The CAR transgenic crossed with the CBA/N mice can be used to determine if adenovirus encoded BTK nucleic acid delivered to stem cells or to peripheral B lymphocytes will cure disease.

Alternatively, a mouse model for scurfy disease (similar to Wiskott-Aldrich) is known in the art (Kanangat et al., Eur. J. Immunol., 1996, 26(1):161-165). A transgene as described herein can be integrated into the germline of the scurfy mouse generating a transgenic mouse that has scurfy disease and whose lymphoid tissue is susceptible to adenovirus infection. The ability of a test gene to treat the disease can be determined by delivering the test gene to lymphoid tissue using a replication defective recombinant adenoviral vector.

### EXAMPLES

The invention is further illustrated by the following examples. The examples are provided for illustration only, and are not to be construed as limiting the scope or content of the invention in any way.

### Example 1: Production of Transgenic Founders

Transgenic mice were produced using a construct in which the gene encoding human CAR (hCAR) was placed under the control of a Major Histocompatibility Complex Class I regulatory sequence (H-2K-i-LTR). This promoter was chosen because of the near ubiquitous and constitutive expression of MHC class I protein on somatic cells, especially cells of hematopoietic origin (Klein, J. (1975) Biology of the mouse histocompatibility-2 complex, Springer-Verlag, New York, Heidelberg and Berlin). Moreover, while the constitutive amount of MHC class I protein can vary greatly between different tissues and among cell types within a tissue, MHC expression can be modulated by cytokines (David-Watine, B., Israel, A. & Kourilsky, P. (1990) Immunol Today 11, 286-292), potentially allowing the upregulation of transgene expression.

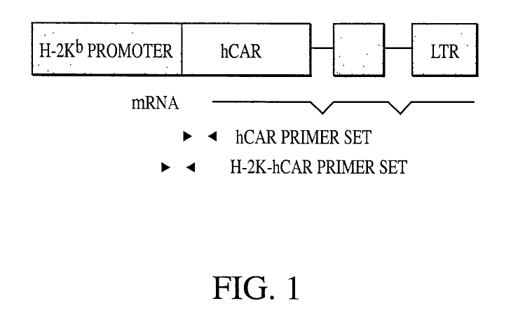

The transgenic mice were generated as follows. A 2.5 kb cDNA sequence encoding hCAR (Bergelson et al., (1997) supra and Bergelson et al., (1998) supra) was cloned into a vector, which has a H-2K-i-LTR expression cassette containing a H-2K promoter and 3′H-2K splice acceptor site (FIG. 1; Domen, J., Gandy, K. L. & Weissman, I. L. (1998) Blood, 91, 2272-82.). Protein expression from the construct was verified by transfection into CAR negative cells. Plasmid DNA was purified using an EndoFree DNA kit (Qiagen, Valencia, Calif.) and the H-2K-hCAR construct was linearized. Transgenic mice were produced on a C57BL/6J (B6) background by standard methods. The MHC-hCAR cassette was excised from a plasmid and microinjected into pronuclei of fertilized B6 oocytes. Injected oocytes were transferred into pseudopregnant female B6 mice and offspring typed for transgene by PCR of DNA from tail snips.

Mice offspring positive for the transgene were identified by PCR of tail DNA prepared using Qiagen DNA prep kits. Two sets of PCR primers were used for typing. One set included sequences from the murine Class I promoter and hCAR and produced a 600 bp fragment: H-2K-hCAR 5′AAAAGCCTCTCTCTCCACTG3′ (SEQ ID NO:1) and 5′ GGCAGTTTCCCCTTTGGCTT3′ (SEQ ID NO:2). A second set of internal primers of hCAR, 5′ CGCTCCTGCTGTGCTTCGTG3′ (SEQ ID NO:3) and 5′ ATCCATCAACGTAACATCTC3′ (SEQ ID NO:4), amplify a 365 bp fragment of hCAR. Neither primer set amplified mCAR sequences from a mCAR cDNA clone or from cDNA prepared from B6 kidney. Five mice positive for the transgene were identified. Four transmitted the transgene and were used to establish founder lines.

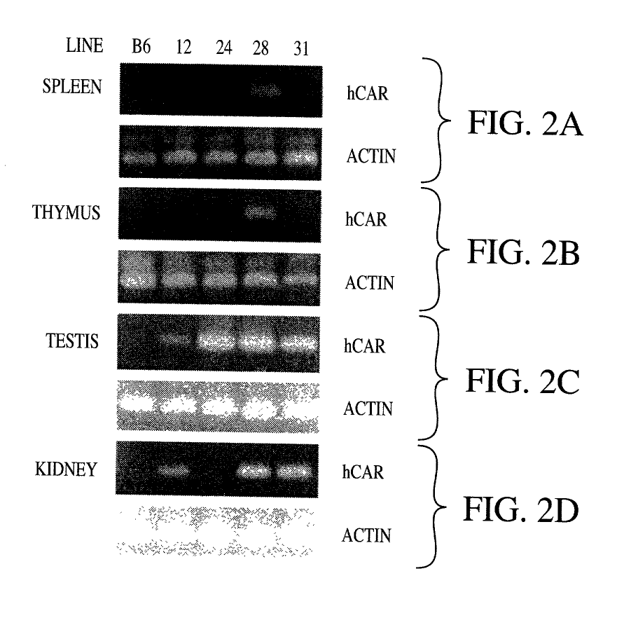

The synthesis of transgene encoded mRNA was determined by RT-PCR of tissues from the transgenic lines. Tissues were analyzed for endogenous expression of mCAR as follows. RNA was extracted from the tissues of transgenic mice and control mice using a Qiagen RNAeasy kit following the manufacturer's instructions. Reverse transcription (RT) reactions were carried out on DNase treated RNA samples using Moloney murine leukemia virus reverse transcriptase, random hexamers (Promega, Madison, Wis.), followed by PCR using the internal hCAR primer set. Reactions where run on a 4% NuSieve agarose gel followed by ethidium bromide staining. Actin was used as a positive control and PCR using primers 5′ ATGGATGACGATATCGCT3′ (SEQ ID NO:5) and 5′ATGAGGTAGTCTGTCAGGT3′ (SEQ ID NO:6) produced a 570 bp fragment. hCAR mRNA was detected in lymphoid tissues (spleen and thymus) from founder line 28 (FIGS. 2A and 2B). There is apparently a tissue-specific restriction because transgene mRNA is readily detected in the testis of all four founder lines whereas only three of the four lines expressed hCAR in the kidney. hCAR mRNA was not detected in any tissue from normal B6 mice despite the fact that kidney cells produce highly homologous mCAR. Since all the tissues express MHC class I protein (David-Watine, supra), regulatory effects exerted on the inserted hCAR transgene by flanking genomic sequences likely account for variable tissue expression.

### Example 2: Transgenic Lymphocytes Express hCAR Protein

Surface staining with RcmB and FACS analysis was used to directly assess hCAR protein expression on lymphoid populations from the transgenic lines. Detection of hCAR, GFP and integrin expression by FACS was performed as follows. Lymphocyte populations from mice that were positive for the transgene were screened for hCAR expression by flow cytofluorimetric analysis. Erythrocyte depleted lymphocyte populations were incubated with a 1:100 dilution of control or RmcB ascites, washed and incubated with anti-IgG₁-FITC, fixed with 2% paraformaldehyde and analyzed by flow cytometry using a either a FACS Calibur or FACS Vantage machine. GFP expression from unfixed cell populations was determined on cells exposed to adenovirus CMV-GFP or adenovirus EF1-GFP and cultured for 48 hours. Concurrent with the GFP analysis lymphoid cell subpopulations were distinguished by staining with phycoerythrin (PE), allophycocyanin (APC) or Cy5PE conjugates of: anti-B220 (CD45R) (RA3-6B2), anti-Thy 1.2 (CD90) (Caltag), anti-CD5 (53-7.3) and anti-Mac 1(CD11b). Integrin expression was analyzed by staining with anti αᵥ-PE. FACS data was analyzed using FlowJo software.

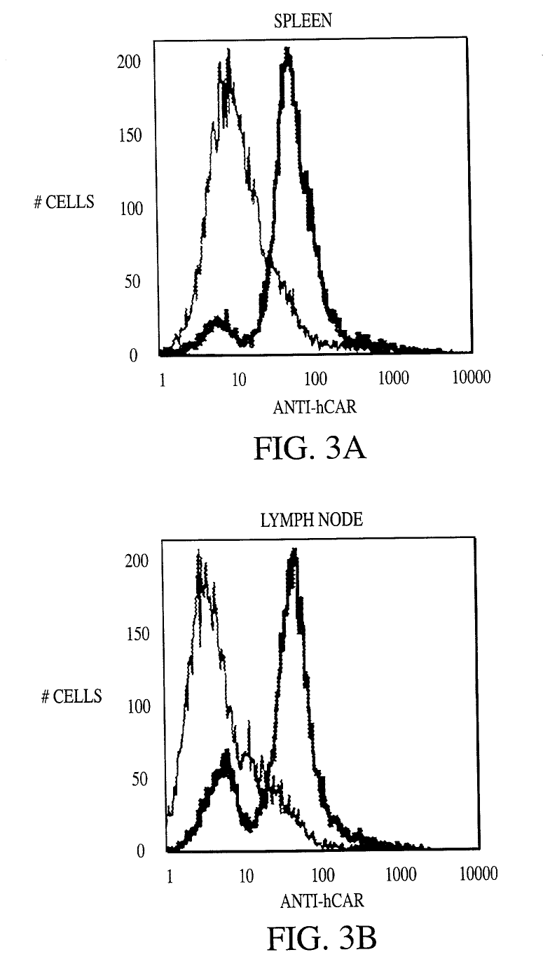

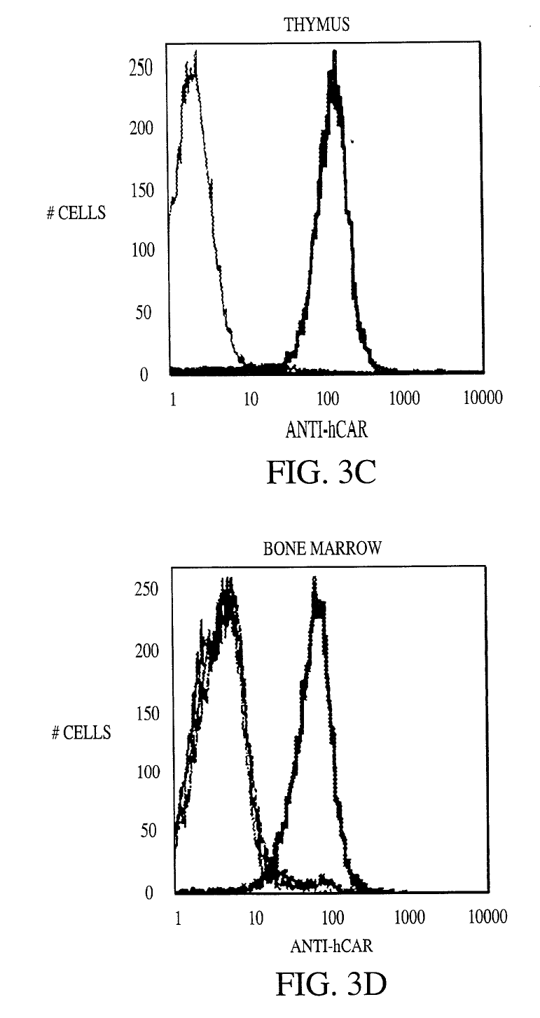

hCAR protein is readily detected on cells from both primary and secondary lymphoid organs of the hCAR⁺ line 28 (FIGS. 3A and 3B). hCAR protein is uniformly expressed on thymocytes and bone marrow cells, whereas, spleen cells and lymph nodes contain a small, and as yet unidentified population of hCAR negative cells in addition to the major hCAR positive population. Consistent with the RT-PCR analysis of mRNA, these same populations taken from normal B6 mice and the three other founder lines did not stain with anti-hCAR antibody.

hCAR cell surface expression was analyzed by immunoprecipitation of biotinylated surface proteins from lymphocyte subpopulations enriched by negative selection. The human kidney cell line, L293A, was used as a source of hCAR for the positive control. Lymphocytes were biotinylated using the cell-impermeable biotin derivative biotinamidocaproic acid 3-sulfo-N-hydroxy-succinaimide ester (Sigma, St. Louis, Mo.) as described by Coligan et al., ((1995) in Current Protocols Immunol., ed. Colco, R. (John Wiley & Sons, Inc., Vol. 2.). Biotinylated lymphocyte populations and control 293 cells were then treated with lysis buffer: 0.5% NP-40, 0.5% DOC, 10 mM Tris pH8.0 plus protease inhibitors, as described in Woodland et al. ((1996) J. Immunol., 156, 2143-2154.). Immunoprecipitation was performed on cell lysates that were precleared with anti-IgG₁ antibody-coupled agarose beads (Sigma, St. Louis, Mo.) that had been incubated with control ascites. Precleared lysates were incubated with anti-hCAR monoclonal antibody, RmcB (Hsu et al., (1988) J. Virol., 62, 1647-1652), which bound to anti-IgG₁ agarose beads as described by Schmidt et al., ((1995) J. Immunol., 155, 2533-2544). Samples were washed and protein eluted from the beads by boiling in SDS-PAGE sample buffer and run on a 10% SDS-PAGE gel. Proteins were electrophoretically transferred to an Immobilon P membrane and developed with Streptavidin-HRP and ECL.

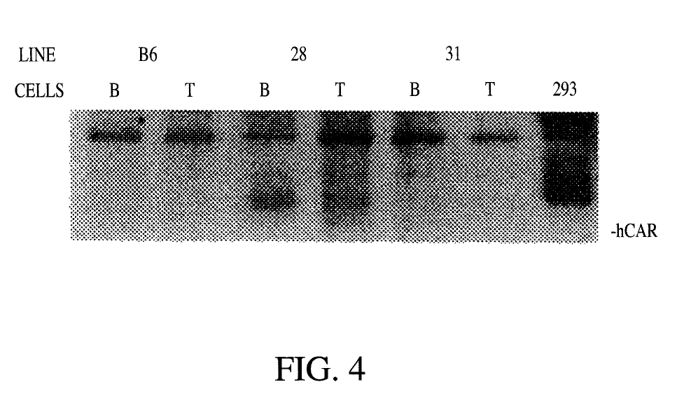

A protein of appropriate size for HCAR, 46 kD, was detected on both purified splenic B and T cells from founder line 28 and L293A cells (FIG. 4). hCAR protein was not detected on splenic lymphocytes from the other founder lines (line 31 shown), or B6 lymphocytes, a finding consistent with the RT-PCR and FACS analyses.

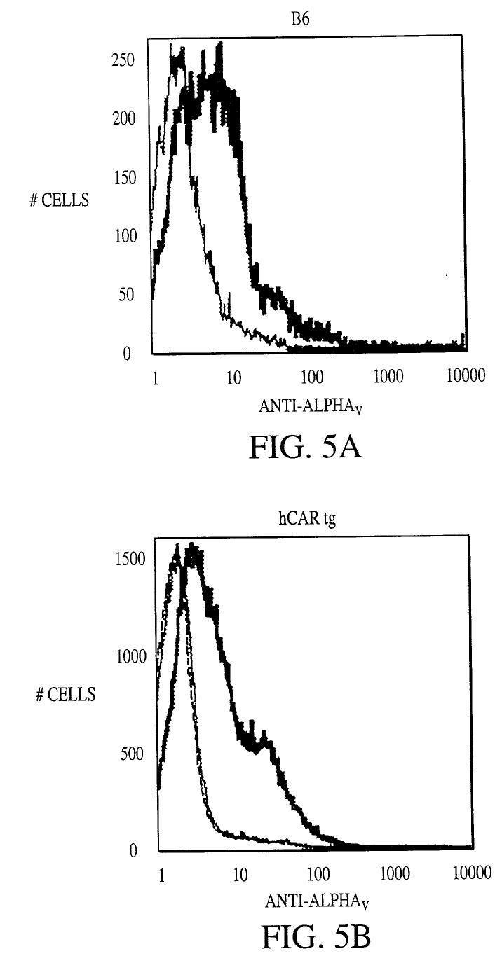

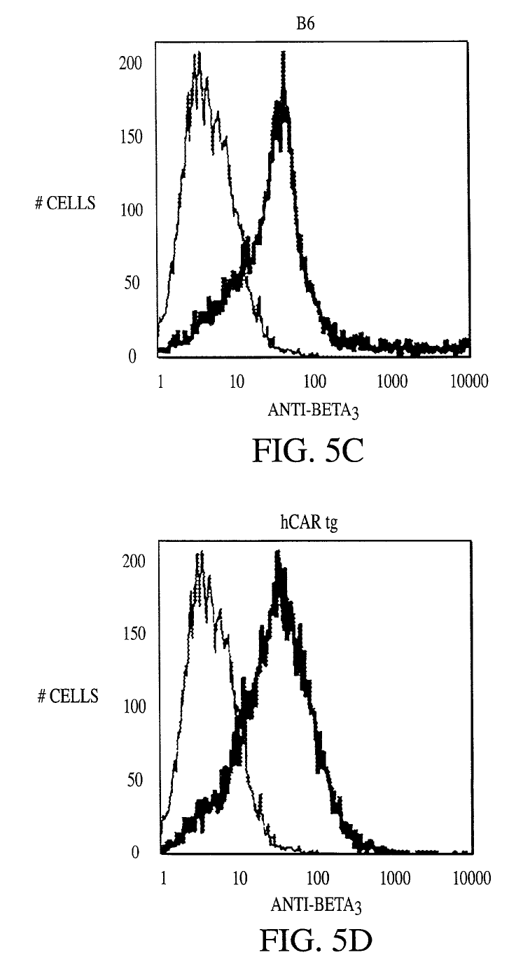

Efficient infection with adenovirus is critically dependent on CAR and facilitated by an integrin co-receptor, αᵥβ₃ or αᵥβ₅, emerow, G. R. & Stewart, P. L. (1999) Micro. Mol. Biol. Rev., 63, 725-734.). FACS analysis was used to assay for integrin because of conflicting reports concerning αᵥ expression on resting lymphocytes (Gerber et al., (1996) Proc. Natl. Acad. Sci., U S. A., 93, 14698-703; Maxfield et al., (1989) J. Expt. Med., 169, 2173-2190; Takahashi et al., (1990) J. Immunol, 145, 4371-4379). FIGS. 5A-B show the surface expression of αᵥ integrin on B6 and hCAR⁺ spleen cells: unstained cells (^{••••}), cells stained with anti-αᵥ-PE (−) or Ig-PE (−−) control antibody. The analysis showed αᵥ integrin is readily detected on lymphocytes from control and transgenic animals, shown here for transgenic and non-transgenic splenic B220⁺ cells.

Integrin β₃ was also detected on resting lymphocytes by FACS analysis. Therefore, the integrin co-receptor that facilitates adenovirus uptake is available for adenovirus infection in lymphocyte populations (See FIGS. 5C and 5D).

### Example 3: Adenovirus Infection and Reporter Expression in hCAR⁺ Lymphocytes

To test whether expression of hCAR renders murine lymphocytes susceptible to adenovirus infection, we prepared cells from spleen, lymph node, thymus, bone marrow, and peritoneal cavity of hCAR line 28 and control B6 mice and exposed them to adenoviral vector containing a CMV immediate/early promoter which is operably linked to a green fluorescent protein (GFP; referred to herein as “adenovirus CMV-GFP”).

Spleen, lymph node, and thymus cell suspensions were prepared as described before (Schmidt et al., supra and Woodland et al., supra), and the cells from three mice were pooled. Briefly, peritoneal cells were obtained by two cycles of lavage with ice cold balanced salt solution (BSS) supplemented with 0.3% BSA and penicillin (10 units/ml), streptomycin (10 μg/ml) and gentamycin (10 μg/ml)(Sigma). Macrophages were depleted from peritoneal washes by incubating the cells for 2 hours at 37° C. on 100 mm tissue culture dishes in complete media. The non-adherent cells were removed by gently washing the plates with cold BSS. In some experiments, resting B cells were prepared by incubating erythrocyte depleted spleen cell populations on anti-CD43 (S7, 5 μg/ml) coated tissue culture plates and T cells on anti-μ (b7.6) coated plates, for 1-2 hrs at 4° C. and harvesting the non-adherent cells in cold BSS. Bone marrow cell suspensions were obtained by flushing femurs with ice cold medium.

Spleen cells, or B cell enriched populations were cultured with B cell activators: 50 μg/ml of E. coli 055:B5 lipopolysaccharide (LPS), or 10 μg/ml anti-Ig plus 100 U/ml of rmIL-4 (Biological Response Modifiers Unit, NIH), or PMA (10 ng/ml) plus Ionomycin (Io, 1 μg/ml). Thymus cells were cultured with 5 μg/ml ConA plus 25 U/ml of rmIL-2 (NIH), and lymph node cells were cultured with the T cell activator 5 μg/ml anti-CD3ε (145-2C11) plus 25 U/ml of rmIL-2. Bone marrow and peritoneal cells were infected as described above and cultured without stimulators.

Adenovirus EF1-GFP is a recombinant adenovirus with GFP expression driven by an EF1α promoter. Lymphocyte suspensions were exposed to adenovirus CMV-GFP, or adenovirus EF1-GFP at a moi=50 for 2 hours at 37° C. in serum-free RPMI-1640 media, washed and treated with neutralizing anti-adenovirus antiserum (Access Biomedical) for 30 minutes at 37° C. to inactivate any input virus not taken up by the cells, washed and cultured in the presence or absence of lymphocyte activators for 48 hours prior to FACS analysis.

After virus exposure, cells were cultured for 48 hours in the presence or absence of B or T cell activators. Thereafter, infected control and transgene positive cell populations were stained for B cell (B220), T cell (Thy 1.2), or macrophage (MAC-1) surface antigens and analyzed by FACS. GFP⁺ lymphocytes were readily detected in all lymphoid tissues from the hCAR⁺ donors but not from B6.

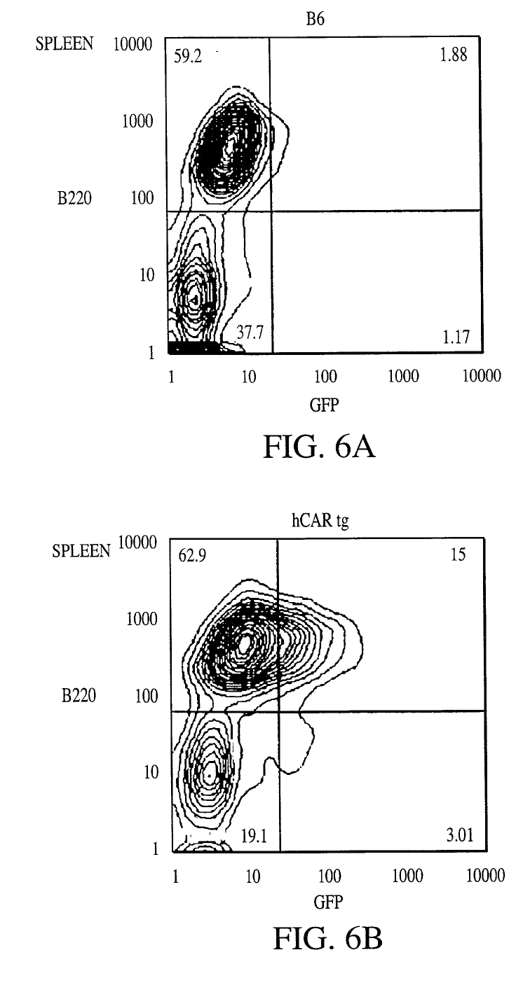

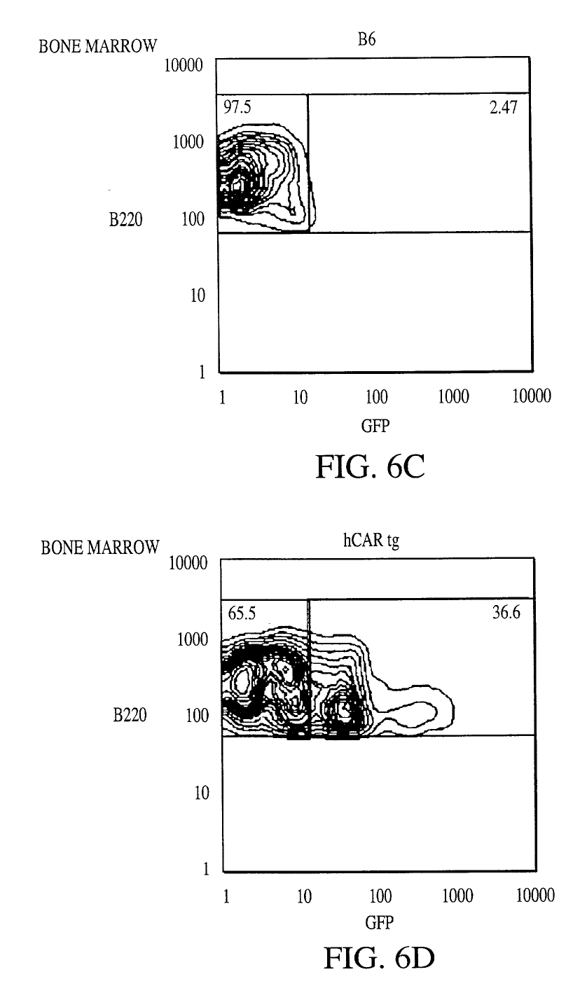

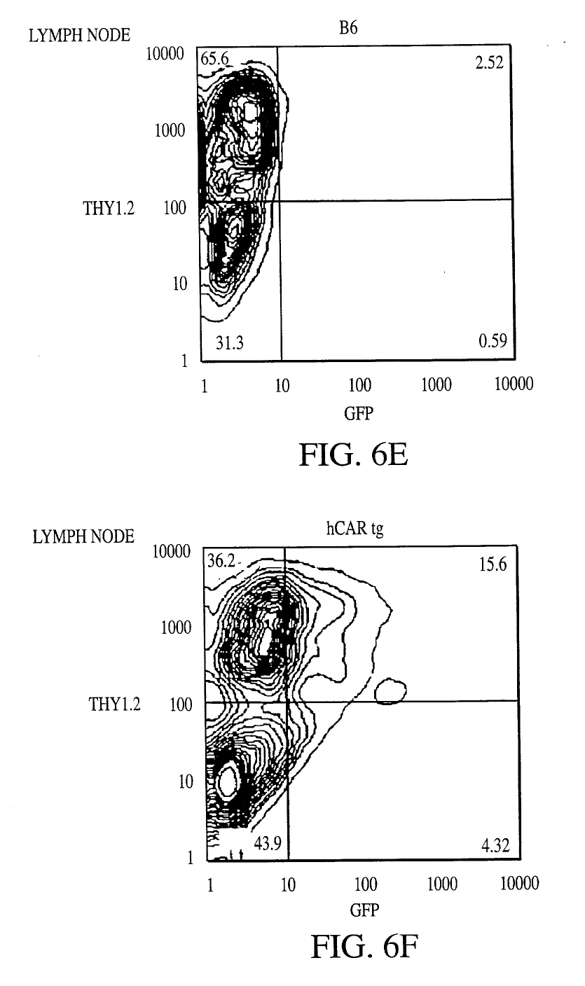

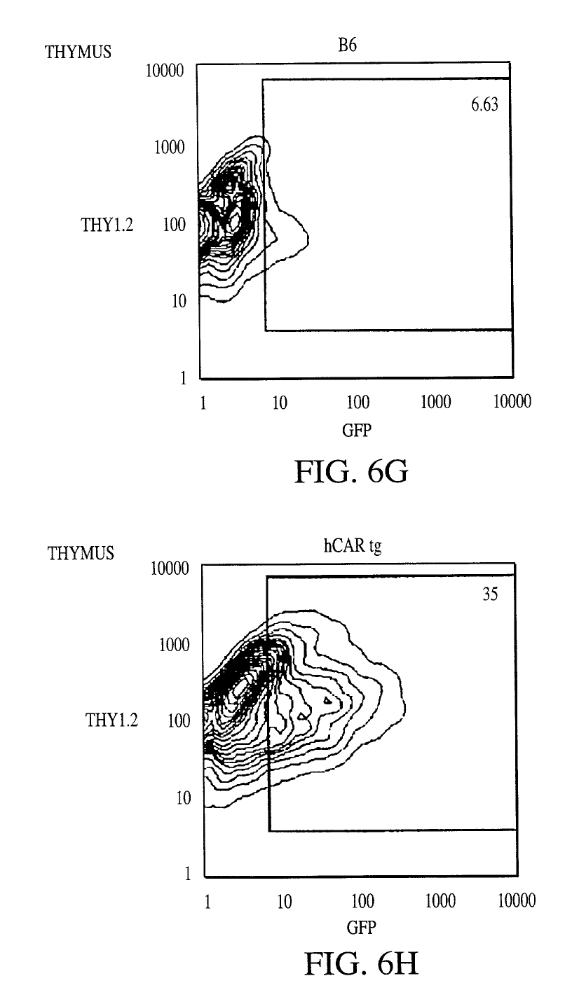

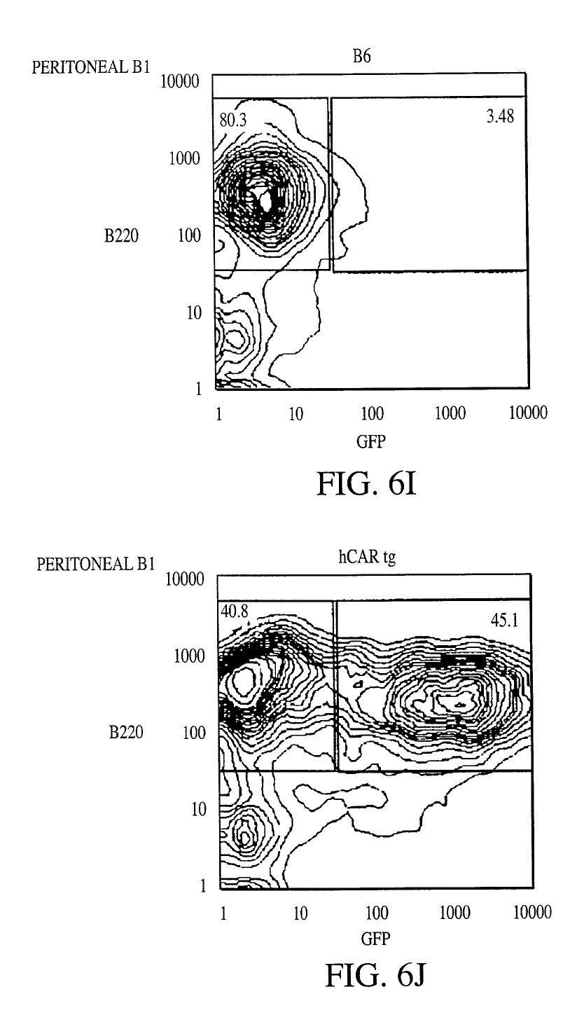

B lymphocytes from spleen (FIGS. 6A and 6B), bone marrow (FIGS. 6C and 6D) and the peritoneal cavity (FIGS. 61 and 6J) are all targets for adenovirus infection. Splenic B220⁺ B cells from the hCAR⁺ mouse, activated for 48 hours with anti-Ig plus IL-4, show a marked increase in GFP¹ cells compared to B6, 15% vs. 1.9% (FIG. 6B). The number of GFP⁺ cells is significantly lower than the number of hCAR⁺ B cells in the spleen, however, and this is likely due to the efficiency of the hCMV promoter. GFP was not detected in unstimulated splenic B cells from either transgenic or normal mice (FIGS. 6A and 6B). Lymphocyte activation per se does not facilitate infection, because wild-type B cells activated before or after adenovirus exposure do not express GFP, although, B6 and line 28 B cells are activated comparably by anti-Ig plus IL-4. The requirement for post-infection activation for GFP expression in B cells was not absolute. B220⁺ B lineage cells from the bone marrow of transgenic mice, cultured without stimulation, demonstrate a high percentage of GFP⁺ cells, 36%, compared to control B6 bone marrow cells, 2.5% (FIGS. 6C and 6B). The highest level of GFP expression and the largest fraction of infected B cells (45%) were found in the unstimulated B1a cell population from the peritoneum (gated on lymphocytes and B220⁺, CD5⁺, Mac1^{lo}) (FIG. 6J).

T cells from the hCAR transgenic can also be infected with adenovirus. As was seen with B cells, T cells required activation post-infection for GFP expression. Lymph node T cells activated with anti-CD3 plus IL-2 demonstrate a marked increase in GFP fluorescence over that seen with mice negative for the transgene T cells, 15.6% vs. 2.5% (FIGS. 6E and 6F). Similarly, thymic T cells, cultured with ConA plus IL-2 support adenovirus infection as evidenced by a large fraction of GFP⁺, Thy 1⁺ cells, 35% (FIGS. 6G and 6H).

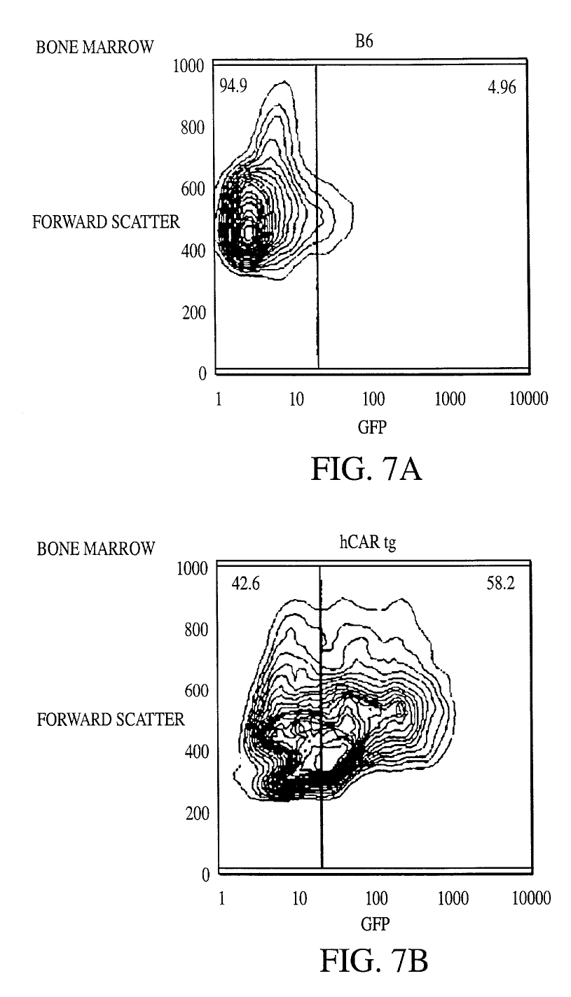

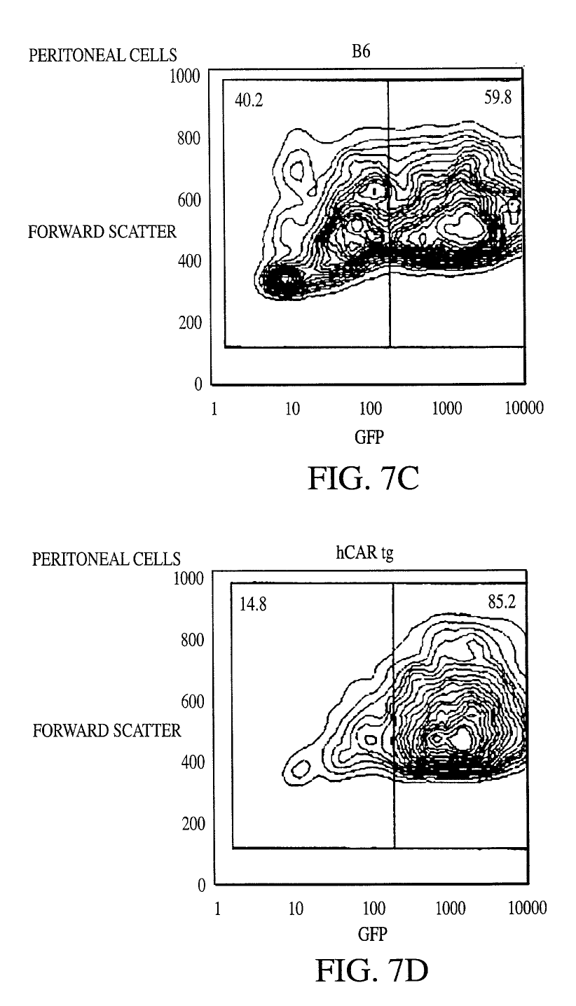

Bone marrow and peritoneal cell populations are heterogeneous tissues containing cell types other than lymphocytes. Because hCAR was expressed on all bone marrow cells, the non-lymphoid cells within this complex population were also tested to see if these cells were susceptible to infection. Bone marrow cells were infected with adenovirus CMV-GFP and cultured without activators for 48 hours. Non-lymphoid cells were identified by electronic gating using forward and side-scatter parameters of populations negative for B220 and Thy1.2. The data in FIG. 7B shows that a high proportion of these cells from hCAR transgene donors (58%) are readily infected by adenovirus CMV-GFP, in contrast, only 5% of a similar population from B6 donors are susceptible to infection (FIG. 7A).

Non-lymphoid peritoneal exudates cells from B6 mice are susceptible to adenovirus infection ( Zhang et al., (1998) Nat Biotech. 16, 1045-9), likely because of endogenous mCAR expression. Although B6 cells were efficiently infected (60%) with adenovirus CMV-GFP (FIG. 7C), hCAR expression potentiated the infection, greater than 85% GFP⁺ (FIG. 7D). This indicates that the presence of the transgene can enhance an efficient infection.

Thus, the findings above show that splenic B2 cells and thymic or lymph node T cells require activation after adenovirus infection in order to express GFP. A number of observations suggest that activation is required to drive gene expression from the vector, rather than changing the susceptibility of lymphoid cells to infection or increasing the protein synthetic capability of small resting lymphocytes. These observations include: (1) Resting B cells express both hCAR and the αᵥβ₃ integrin necessary for adenovirus attachment, uptake and uncoating; (2) The level of GFP expression is dependent on the promoter used, not on cell activation; (3) EF1α is more efficient than CMV, and stimulation with LPS, which can induce cells to proliferate and differentiate to antibody secretion, fails to induce GFP expression; (4) Peritoneal B1a cells, a subpopulation of self-renewing B cells, exhibited the highest level of GFP after infection and the highest proportion of infected cells without needing in vitro activation; (5) A population of bone marrow B or B progenitor cells also supports GFP synthesis without stimulation; and (6) B6 lymphocytes remained refractory to adenovirus infection even after activation.

Taken together, the data minimize the requirement for an activation induced increase in cellular biosynthetic machinery and are more consistent with the notion that resting primary B2 and T cells lack the transcription factors necessary to drive the hCMV and EF1α promoters used in the adenovirus constructs. The finding is supported by the fact that transgenic mice infected with an adenovirus which contains the hCMV immediate/early promoter operably linked to GFP did not express GFP in all tissues or cells of transgenic mice, however, GFP expression could be enhanced when the cells were appropriately stimulated. Similarly, it has been shown that PKC activators enhance the constitutive reporter levels of hCMV driven genes in lymphoid cell lines. This is consistent with the above results showing GFP induction with the PKC activators anti-Ig and PMA.

Example 4: Regulatory Sequences Modulate Reporter Expression in Infected B Cells

Follicular splenic (B2) B cells require post-infection activation for GFP expression while peritoneal B1 cells do not. One reason for this may be that the transcription factors necessary for activating the CMV promoter are not available in resting B2 cells. Alternatively, small resting B cells may lack an adequate protein synthetic capability necessary for efficient expression of the reporter. In this regard, it is noteworthy that B1 cells are more biosynthetically active and larger than resting B2 cells. To assess these possibilities, resting splenic B cells were infected with different adenovirus constructs having GFP driven by different promoters (CMV or EF1α). We also stimulated infected cells with anti-Ig+IL-4, LPS, or PMA+Io as these activators cause cell enlargement and drive B cell proliferation while mobilizing both overlapping and unique transcription factors (Chiles et al., (1992) J. Immunol, 149, 825-831; Huo et al., (1995) J. Immunol, 154, 3300-3309; Francis et al., (1998) Int. Immunol, 10, 285-293; Klaus et al., (1986) Eur. J. Immunol, 16, 92-97).

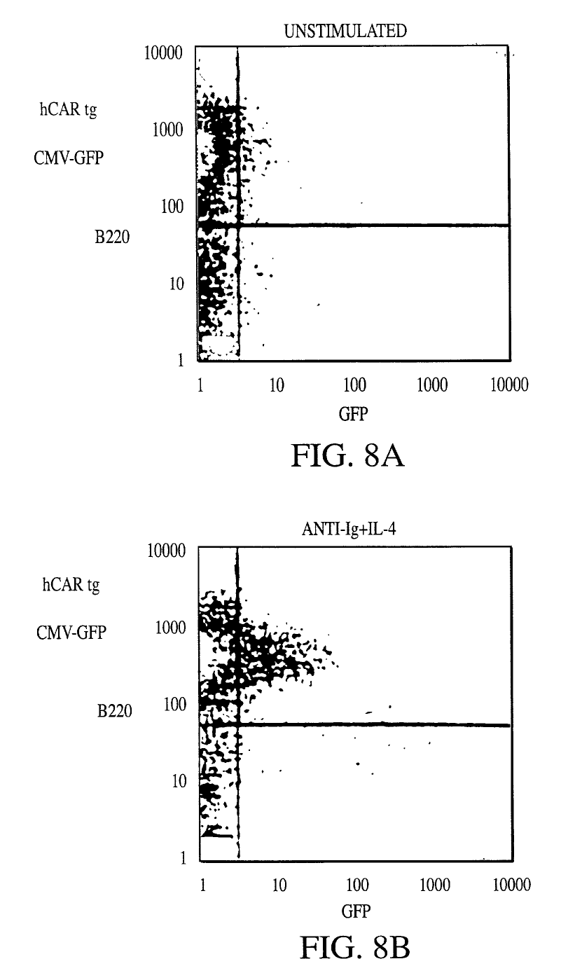

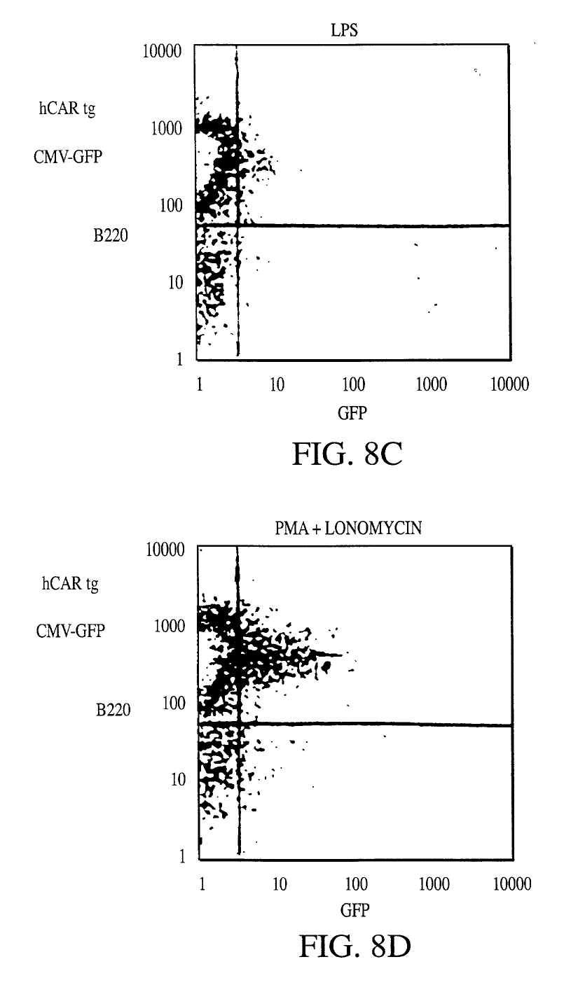

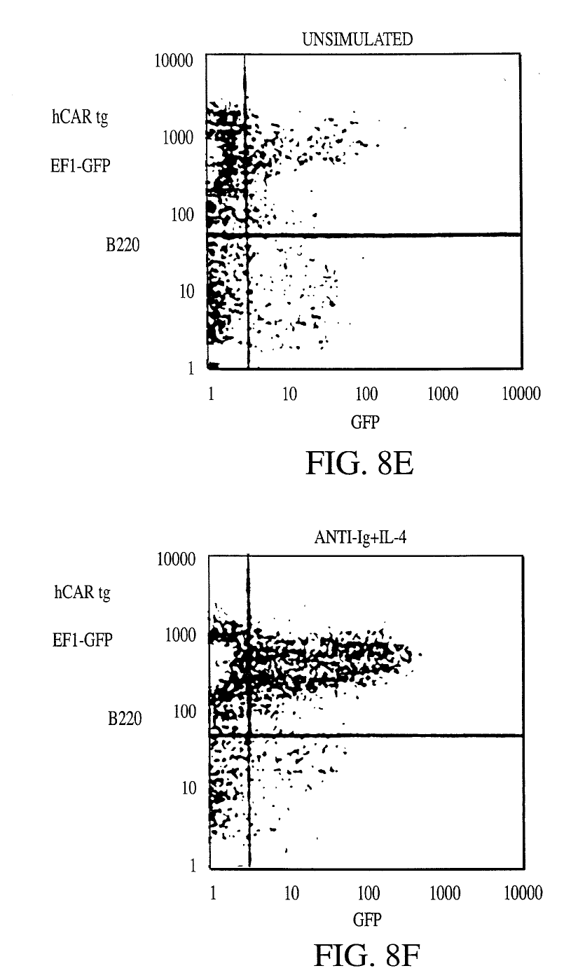

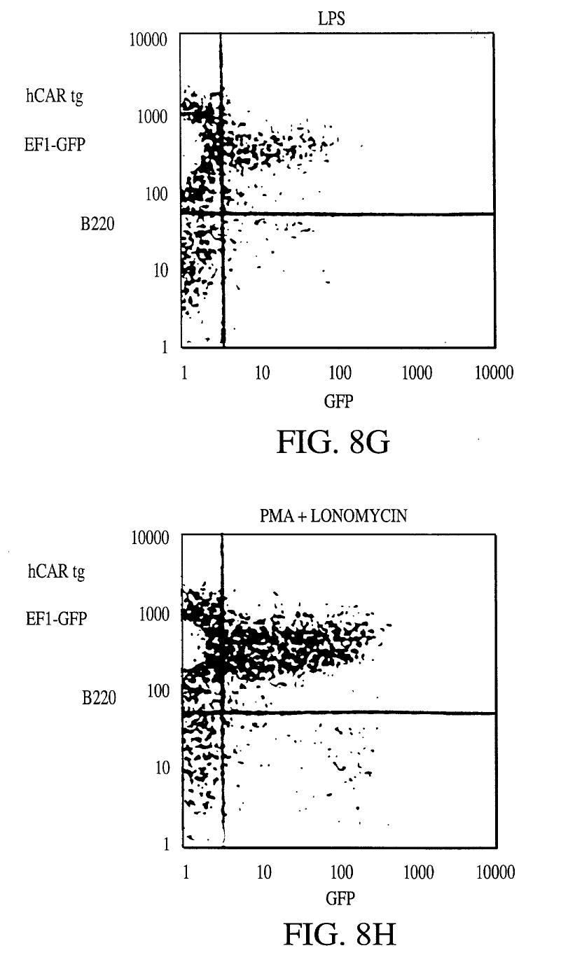

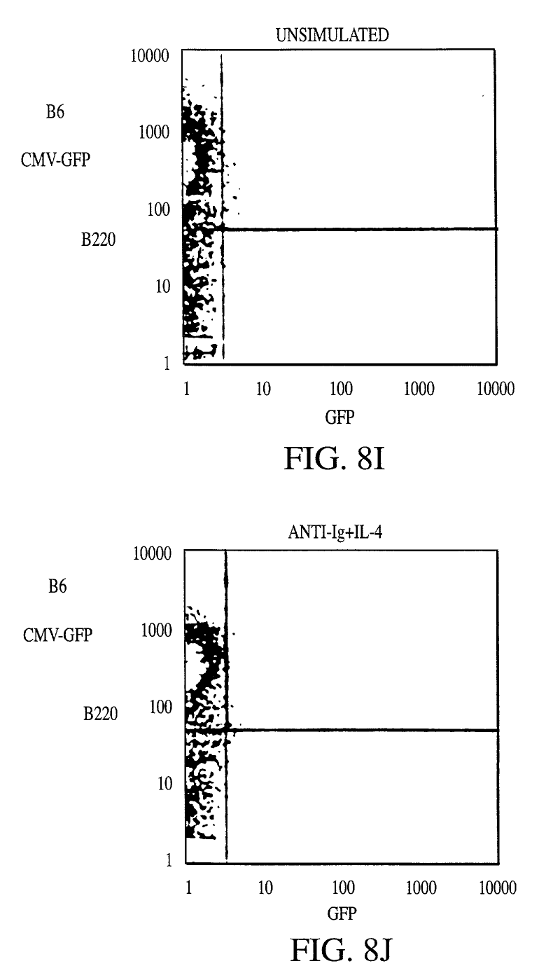

A number of points emerge from this analysis. GFP expression is promoter dependent, as it is markedly enhanced in anti-Ig+IL-4 stimulated hCAR⁺ B cells infected with adenovirus EF1-GFP (FIG. 8F), compared to adenovirus CMV-GFP infected B cells (FIG. 8B). More B cells express GFP (37 vs 12%) and the level of GFP is higher (geometric mean intensity: 22 vs 10 fluorescence units). PMA+Io is similarly effective (FIG. 8D and 8H). Although, GFP expression in unstimulated infected B cells is low, more GFP⁺ cells are found in the population infected with the adenovirus EF1-GFP construct (6 vs 2%; FIG. 8A and 8E). These promoter dependent differences in GFP expression were seen only in infected primary B2 cells. Unstimulated peritoneal B1a cells infected with adenovirus CMV-GFP or adenovirus EF1-GFP showed a high frequency of GFP⁺ cells (37 and 35%) with high levels of GFP expression (geometric mean 125 and 137 fluorescent units). Moreover, high and equivalent levels of GFP were produced by both constructs in infected L293A cells. As before, B6 B cells did not express GFP even after activation (shown for adenovirus CMV-GFP). Finally, B cell activation per se is not sufficient for reporter expression, because LPS fails to induce GFP in B cells infected with either the adenovirus CMV-GFP or adenovirus EF1-GFP construct. Neither the frequency nor signal intensity of GFP⁺ cells is significantly increased over that seen in unstimulated infected B cells, despite the fact that both cell cycle and ³H-thymidine incorporation determinations show LPS is as potent an activator of B cells, as are anti-Ig+IL-4 or PMA+Io.

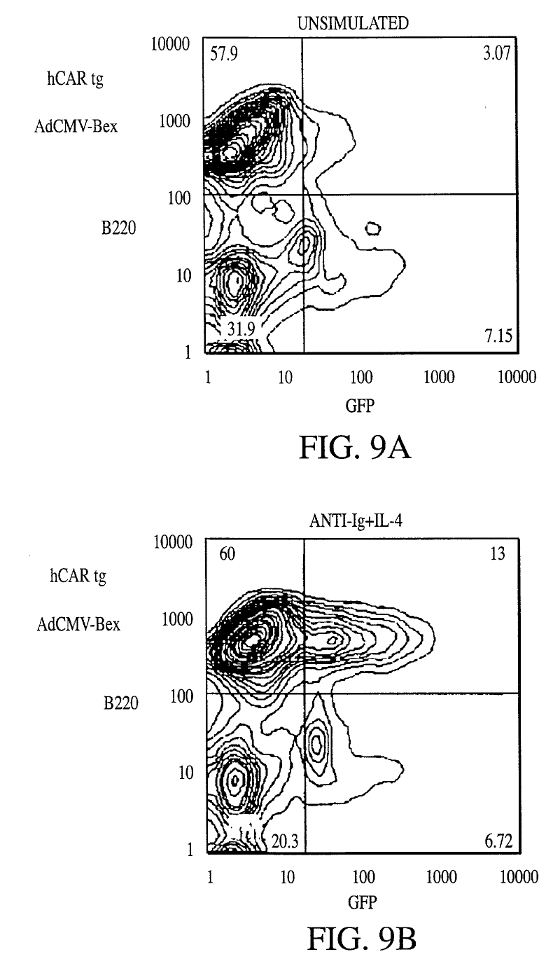

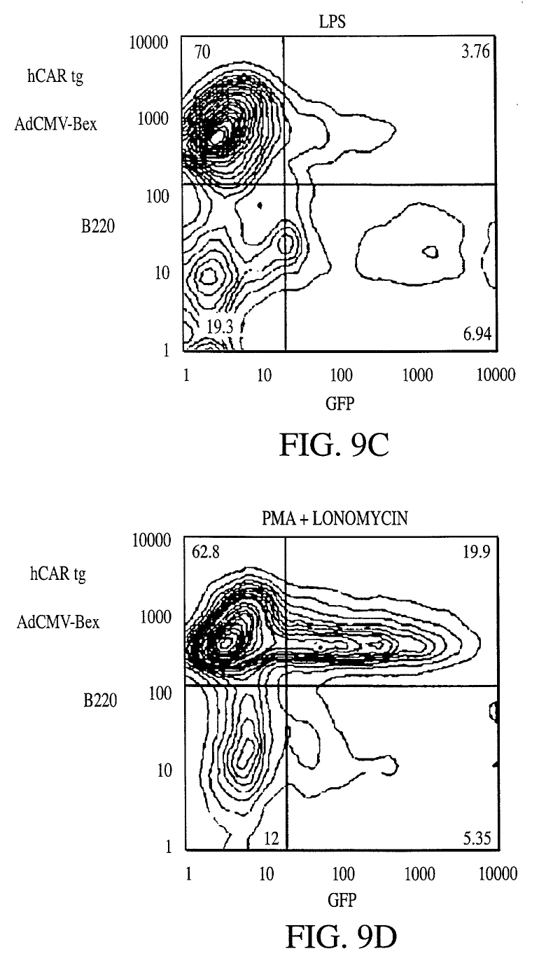

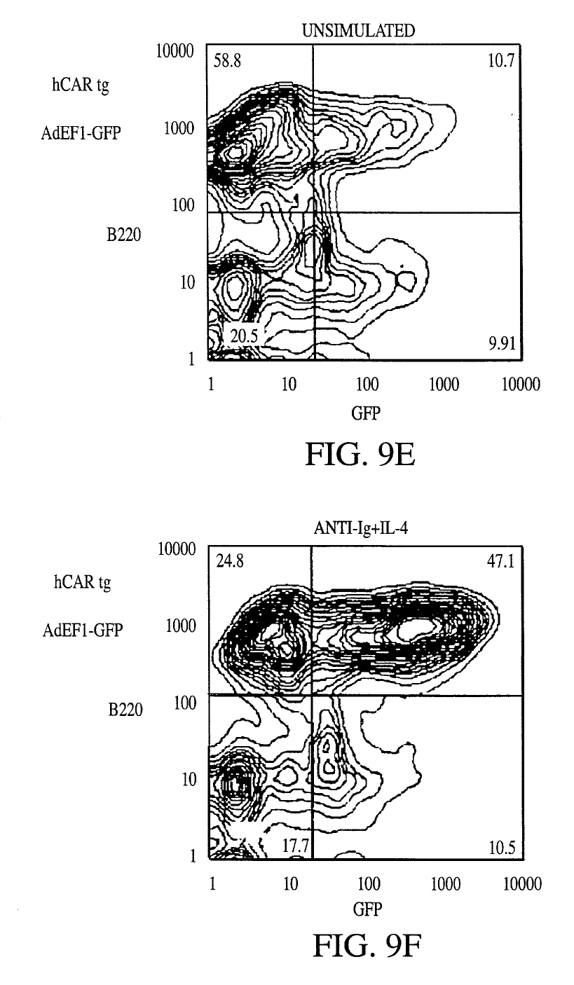

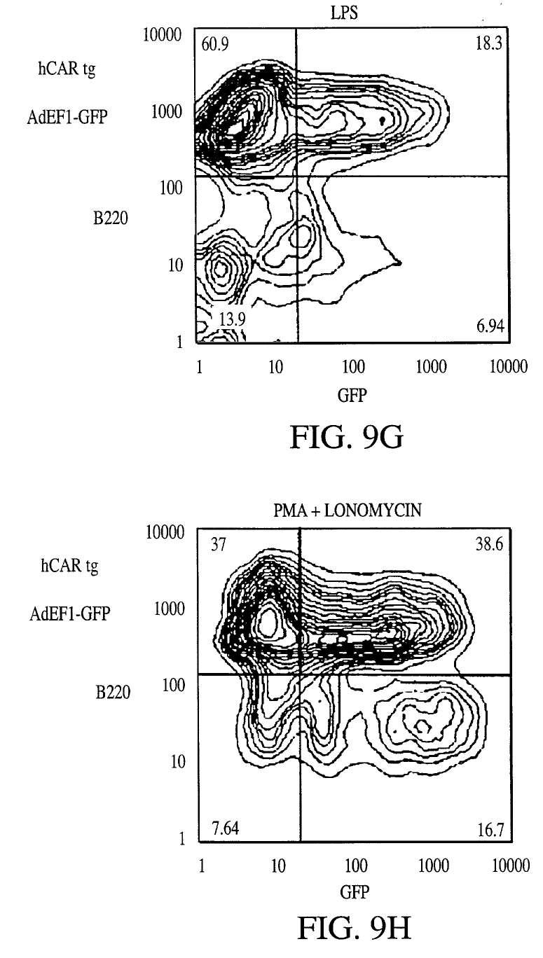

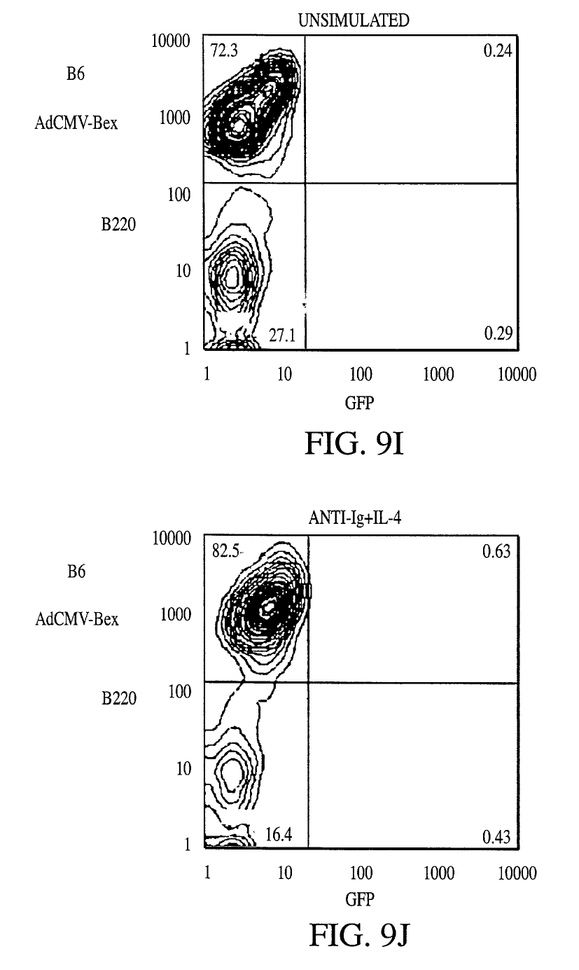

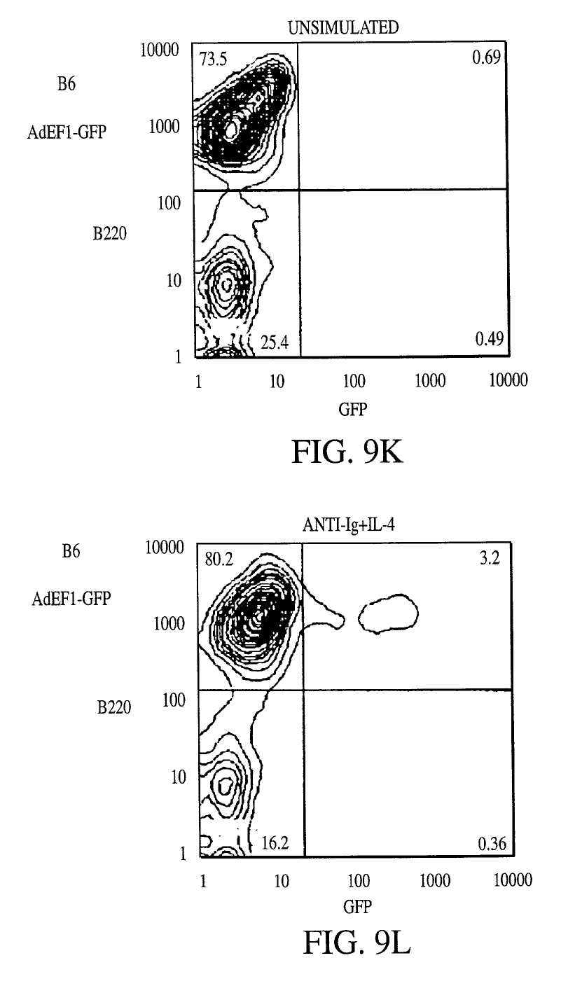

FIGS. 9A-L illustrate the results of a repeat of the experiment shown in FIGS. 8A-J with the modification that BEX, a mutated and brighter version of GFP, driven by the same CMV regulatory sequences was used for the infection. Moreover, the FACS profiles in FIGS. 9A-L were generated using a different mode of data compensation, which resulted in better signal to noise discrimination. These data show, as was found previously, the same hierarchy in the ability of the activators used to drive reporter expression from the constructs; the EF1a construct was more responsive to induction than was the CMV construct.

Thus, the peritoneal B1a subpopulation is readily infected and efficiently expresses GFP. B1 cells are considered resting by numerous criteria, however, they are capable of self-renewal, and unlike B2 cells, they have STAT-3 mobilized to the nucleus. These characteristics support the possibility that differences in important and as yet unidentified transcription factors exist between the B1 a and B2 subpopulations. More importantly, their ability to self-renew, to support high levels of reporter expression, and their sequestered anatomical location may make peritoneal B1a cells ideal targets for long term expression of adenovirus delivered genes.

### OTHER EMBODIMENTS

Other embodiments are within the following claims.

What is claimed is:

1. A transgenic, non-human animal, one or more of whose cells comprise a transgene comprising a regulatory sequence operably linked to a nucleic acid sequence encoding a Coxsackie/adenovirus receptor (CAR), wherein the transgene is expressed in one or more cells of the transgenic animal resulting in the animal exhibiting increased susceptibility to adenovirus infection as compared to a wildtype animal.

2. The animal of claim 1, wherein the transgene is expressed in one or more hematopoietic cells.

3. The animal of claim 1, wherein the transgene is expressed in one or more lymphocytes.

4. The animal of claim 1, wherein the animal develops or has a disorder.

5. The animal of claim 1, wherein the animal has an immunodeficiency disorder.

6. The animal of claim 5, wherein the immunodeficiency disorder is X-linked severe combined immune deficiency, X-linked agammaglobulinemia, or Wiskott-Aldrich syndrome.

7. The animal of claim 1, wherein the regulatory sequence is constitutive or inducible.

8. The animal of claim 1, wherein the regulatory sequence is an MHC class I promoter.

9. The animal of claim 1, wherein the regulatory sequence is an exogenous sequence.

10. The animal of claim 9, wherein the animal comprises one or more cells that comprise an endogenous regulatory sequence that controls the transcription of an endogenous CAR, and wherein the exogenous regulatory sequence controlling transcription of a CAR is different from the endogenous regulatory sequence controlling the transcription of the endogenous CAR.

11. The animal of claim 1, wherein the nucleic acid sequence is substantially identical to a nucleic acid sequence encoding a mouse or human CAR.

12. The animal of claim 1, wherein the animal is a rodent.

13. The animal of claim 1, wherein the animal is a mouse.

14. An isolated nucleic acid molecule comprising an MHC class 1 regulatory sequence operably linked to a nucleic acid sequence that encodes a CAR.

15. The nucleic acid sequence of claim 14, wherein the nucleic acid sequence encodes a human or mouse CAR.

16. A vector comprising the nucleic acid sequence of claim 14.

17. The vector of claim 16, wherein the vector is a plasmid or a viral vector.

18. A cell containing the nucleic acid sequence of claim 14.

19. A cell derived from the animal of claim 1.

20. The cell of claim 19, wherein the cell is a lymphocyte, a liver cell, or a spleen cell.

21. A method of infecting a cell with an adenovirus, the method comprising contacting a cell engineered to express a nucleic acid sequence encoding a Coxsackie-adenovirus receptor (CAR) with an adenovirus such that the adenovirus infects the cell.

22. The method of claim 21, wherein the cell is normally refractory to adenovirus infection.

23. The method of claims 21, wherein the cell is in an animal.

24. A method of determining whether an adenoviral vector infects a cell in vivo, the method comprising:
(a) contacting a cell from the transgenic animal of claim 1 in vivo with an adenoviral vector comprising a test gene encoding a test protein; and
(b) determining expression of the test gene in the cell, wherein expression of the test gene in the cell indicates that the cell can be infected with the adenoviral vector.

25. The method of claim 24, wherein the cell is normally refractory to adenoviral vector infection.

26. A method of testing the efficiency of a regulatory sequence to express a nucleic acid sequence in a cell in vivo, the method comprising:
(a) contacting a cell engineered to express a nucleic acid sequence encoding a Coxsackie/adenovirus receptor (CAR) in vivo with an adenoviral vector comprising a test regulatory sequence operably linked to a reporter gene, and
(b) determining the level of expression of the reporter gene in the cell, wherein the level of expression is an indication of the efficiency of the regulatory sequence.

27. The method of claim 26, wherein the cell is a tumor cell.

28. The method of claim 26, wherein the tumor cell is a prostate tumor cell.
